(19) United States
(12) Patent Application Publication
(10) Pub. No.: US 2017/0312547 A1
(43) Pub. Date: Nov. 2, 2017

(54) METHOD TO RECONSTRUCT THE 3D MAP OF THE RADIATION TREATMENT ISOCENTER OF A MEDICAL ACCELERATOR

(71) Applicant: THE JOHNS HOPKINS UNIVERSITY, Baltimore, MD (US)

(72) Inventors: John Wong, Towson, MD (US); Kai Ding, Baltimore, MD (US); Kang-Hsin Wang, Laurel, MD (US)

(21) Appl. No.: 15/583,065

(22) Filed: May 1, 2017

Related U.S. Application Data
(60) Provisional application No. 62/329,258, filed on Apr. 29, 2016.

(51) Int. Cl. A61N 5/10; H05H 15/00

(52) U.S. Cl. CPC A61N 5/1075; H05H 15/00; A61N 5/1082; A61N 2005/1074

(57) ABSTRACT

For the delivery of high precision radiation treatment, the accuracy with which a target is irradiated at individual gantry, collimator and patient couch orientation is traditionally verified in 2D. With the QA device described herein, the coverage of the gantry is uniquely measured in 3D. The method of the present invention, combining with the collimator and patient couch measurements, allows the reconstruction of the target coverage in full 3D, which was not possible before. In addition, the method of the present invention can be applied to decompose the traditional quality assurance measurements of combined gantry, collimator and patient couch orientations with standard devices. Such an application provides a comprehensive description of the irradiation accuracy.

### CROSS REFERENCE TO RELATED APPLICATION

This application claims the benefit of U.S. Provisional Patent Application No. 62/329,258 filed on Apr. 29, 2016, which is incorporated by reference, herein, in its entirety.

### FIELD OF THE INVENTION

The present invention relates generally to radiation therapy. More particularly the present invention relates to a method to reconstruct the 3D map of the radiation treatment isocenter of a medical accelerator.

### BACKGROUND OF THE INVENTION

Medical accelerators are used for radiation treatment of cancer patients and emit radiation in 360°. A patient lies on a platform, while a head of the device is rotated around the body of the patient. Quality assurance (QA) of the integrity of the medical accelerators is paramount to ensure the safe delivery of radiation treatment. The two main criteria used for QA is radiation reproducibility and mechanical integrity of the machine. The QA tasks specifically consist of quantifying the accuracy and precision of mechanical motions of the accelerators, various optical indicators, and the delivered dosimetry. FIG. 1 illustrates a chart of QA tasks per medical accelerator that must be performed daily, monthly, and annually.

FIG. 2 illustrates a front view of a medical accelerator used for radiation treatment. As illustrated in FIG. 2, one task of QA is to ensure that radiation beams along an axis and the positioning aids, in the form of laser beams, align at an isocenter of the medical accelerator. QA of this alignment is important in order to verify that the radiation beams along axis 1 are configured to treat the patient accurately without causing burns or other undesired side effects.

At present, almost all of the QA tasks listed in FIG. 1 are performed with different apparatus. The mechanical and optical components are examined visually, where the data is not amenable to documentation. For example, a technician checks the light source by directing the source onto the patient couch, and rotating it to make sure it moves about the isocenter of the medical accelerator. The dosimetry is measured with a variety of ionization detectors with the two-dimensional array of discrete detectors being most popular. Higher resolution measurements are measured with films where the data conversion process can be tedious. Some of these tasks are performed on a daily, monthly or yearly basis, and can take hours to perform. For example, while daily maintenance only takes approximately 10 minutes, a monthly maintenance of such machines to test the output and the mechanical integrity of the machine typically will take between 5-6 hours, the results of the testing partially documented. In fact, annual maintenance on the machine can take approximately 4 days to complete.

Currently, to assess integrity of collimator rotation, a piece of film is placed flat on the couch at the plane of the isocenter. A long slit beam of narrow width is set up, such as a slit of approximately 0.1 to 1 cm×20 cm. The piece of film is exposed with the collimator rotated at different angles. Alternately, with the collimator fixed, the table can be rotated at different angles. The film is developed after several different angles are shot to determine, visually or digitally, the dimension of the circle that encompasses all of the intersections of horizontal rays. This gives a measure of the isocenter integrity of collimator rotation. The resultant film is often referred to as a star-shot image. Alternatively, the film can be replaced with a digital device, such as a two-dimensional diode array, an electronic portal imaging device or a flat panel imager; some of which are placed on the table, others can be mounted on the gantry downstream of the table at an orthogonal beam's eye view of the radiation to receive a projection image of the radiation slit.

The current method to assess the integrity of gantry rotation includes sandwiching a vertical film between two blocks of plastic. The sandwich of blocks and film is placed vertically in the plane of gantry rotation. A long slit beam is set up with the narrow width orthogonal to the vertical film. The gantry is rotated 360° around the vertical film block exposing the film at different gantry angles. After several different angles are shot, the film is developed. The dimension of the circle that encompasses all of the intersections of the vertical rays is determined either visually or digitally. This gives a measure of the isocenter integrity of gantry rotation. The two dimensional electronic detectors that can be used for collimator rotation measurements are not applied for this application because of poor detector resolution or concern for radiation damage,

Accordingly, there is a need in the art for a device that provides a faster and more accurate measurement of collimator rotation and gantry rotation.

### SUMMARY

According to a first aspect of the present invention a method for real-time mechanical and dosimetric quality assurance measurements in radiation therapy includes obtaining measurements of an orientation of a collimator and patient couch of the medical accelerator in two dimensions using a QA device at an isocenter of a medical accelerator. The method includes measuring coverage of the gantry in three dimensions using a QA device at an isocenter of a medical accelerator. The method also includes reconstructing target coverage of the gantry in three dimensions.

In accordance with an aspect of the present invention, wherein the component of the medical accelerator includes the collimator, the table/couch, and the gantry. The method includes rotating the QA device about a treatment isocenter of the medical accelerator. The method includes rotating the QA device orthogonal to a gantry beam such that a transformation can be performed on the projected beam image. Additionally, the method includes providing a piercing point of the gantry beam in (x, y, z) or three dimensional coordinates. The method includes displaying results of the measurements taken with the QA device. The method also includesdisplaying errors in alignment of components of the medical accelerator based on the measurements taken with the QA device.

In accordance with an aspect of the present invention, a method for verifying a patient's radiosurgery, includes securing an extended bb marker off of a treatment couch in a treatment room. The method includes irradiating the extended bb marker with a treatment beam from a medical accelerator and imaging the irradiation with a flat panel imager. The method also includes verifying that the treatment beam covers the bb for all combinations of orientation of the medical accelerator and reconstructing and displaying an image of any combination of rotation of a component of the medical accelerator.

In accordance with another aspect of the present invention, the method includes irradiating the extended bb marker at a number of orientations of a gantry, collimator, and couch of the medical accelerator. The method includes performing 15 to 20 irradiations of the extended bb marker at different orientations of the gantry, collimator, and couch of the medical accelerator. The method includes calibrating the medical accelerator based on the outcome of the irradiation. The method includes positioning the flat panel imager on a gantry on an opposite side of the accelerator head. The method also includes reconstructing an image of a gantry, collimator, and couch of the medical accelerator. The method also includes calculating displacement between the actual beam center and an idealized “point” radiation isocenter.

In accordance with an aspect of the present invention, a method for real-time mechanical and dosimetric quality assurance measurements in radiation therapy, includes obtaining measurements of an orientation of a component of the medical accelerator in two dimensions using a quality assurance (QA) device at an isocenter of a medical accelerator. The method includes measuring coverage of the gantry in three dimensions using a QA device at an isocenter of a medical accelerator. The method also includes reconstructing target coverage of the gantry in three dimensions. Additionally, the method includes calculating displacement between the actual beam center and an idealized “point” radiation isocenter.

In accordance with another aspect of the present invention, the method includes irradiating the QA device at a number of orientations of a gantry, collimator, and couch of the medical accelerator. The method includes performing 15 to 20 irradiations of the QA device at different orientations of the gantry, collimator, and couch of the medical accelerator. Additionally, the method includes calibrating the medical accelerator based on the outcome of the irradiating. The method includes positioning the QA device on a gantry on an opposite side of the accelerator head. The method also includes reconstructing and displaying an image of a gantry, collimator, and couch of the medical accelerator.

### BRIEF DESCRIPTION OF THE DRAWINGS

The accompanying drawings provide visual representations which will be used to more fully describe the representative embodiments disclosed herein and can be used by those skilled in the art to better understand them and their inherent advantages. In these drawings, like reference numerals identify corresponding elements and:

FIG. 1 illustrates a chart of QA tasks per medical accelerator that must be performed daily, monthly, and annually.

FIG. 2 illustrates a front view of a medical accelerator used for radiation treatment.

FIG. 3A illustrates a perspective view of a device according to the features of the present invention sitting within the treatment range of a medical accelerator. FIG. 3B illustrates a perspective view of a device according to the features of the present invention sitting within the treatment range of a medical accelerator. FIG. 3C illustrates a perspective view of a device according to the features of the present invention sitting within the treatment range of a medical accelerator. FIG. 3D illustrates a perspective view of a device according to the features of the present invention sitting within the treatment range of a medical accelerator.

FIG. 4A illustrates a partially top-down perspective view of a device for real-time mechanical and dosimetric quality assurance measurements in radiation therapy according to the features of the present invention. FIG. 4B illustrates a perspective view of a device for real-time mechanical and dosimetric quality assurance measurements in radiation therapy according to the features of the present invention. FIG. 4C illustrates a perspective view of a rotated device for real-time mechanical and dosimetric quality assurance measurements in radiation therapy according to the features of the present invention.

FIG. 5A illustrates a partially sectional perspective view of a device for real-time mechanical and dosimetric quality assurance measurements in radiation therapy according to the features of the present invention. FIG. 5B illustrates a partially sectional perspective view of a device for real-time mechanical and dosimetric quality assurance measurements in radiation therapy according to the features of the present invention. FIG. 5C illustrates a partially sectional perspective view of a device for real-time mechanical and dosimetric quality assurance measurements in radiation therapy according to the features of the present invention. FIG. 5D illustrates a partially sectional perspective view of a device for real-time mechanical and dosimetric quality assurance measurements in radiation therapy according to the features of the present invention.

FIG. 6 illustrates a side view of a QA device according to an embodiment of the present invention.

FIG. 7 illustrates a diagram of a method according to the features of the invention.

FIGS. 8A and 8B illustrate schematic diagrams showing a method according to the features of the invention. Note that the laser image has never been captured with prior art of QA devices.

FIG. 8C illustrates a method of performing table positioning QA, according to an embodiment of the present invention. Note that the light field image with the optical distant indicator has never been captured with prior art of QA devices.

FIG. 8D illustrates a method of performing rotation QA as captured optically for documentation, according to an embodiment of the present invention.

FIG. 8E illustrates a method of performing optical and radiation coincidence of a field shaped by the multi-leaf collimator.

FIG. 9 illustrates a hard coded digital image scale, according to an embodiment of the present invention.

FIG. 10 illustrates an analysis tool, provided by the software control program for the device of the present invention.

FIG. 11 illustrates a localization tool, provided by the software control program for the device of the present invention.

FIG. 12 illustrates a region of interest tool, provided by the software program for the device of the present invention.

FIG. 13 illustrates an exemplary screen for defining scale in an acquired image taken using the device of the present invention.

FIG. 14A illustrates an example of a program feature to superimpose images taken with the device of the present invention.

FIG. 14B illustrates an example of an image for analysis of co-linearity of the laser taken using the device of the present invention.

FIG. 15 illustrates an image of an exemplary recording of laser QA tasks executed with the device of the present invention.

FIG. 16A illustrates an image of an exemplary recording of laser QA tasks executed with the device of the present invention. FIG. 16B illustrates an image of an exemplary embodiment of the user interface showing the acquisition of a right room laser QA.

FIG. 17 illustrates an image of an exemplary recording of laser QA tasks executed with the device of the present invention.

FIG. 18 illustrates an image of an exemplary recording of laser QA tasks executed with the device of the present invention.

FIG. 19 illustrates laser alignment analysis using the device of the present invention.

FIG. 20 illustrates laser alignment analysis using the device of the present invention.

FIGS. 21A-F, 22A-F, and 23A-C illustrate exemplary recordings of table movement and optical distance indicator (ODI) QA tasks executed with the device of the present invention. More particularly, FIGS. 21A-F relate to QA for table vertical movements captured using the device of the present invention, FIGS. 22A-F relate to QA for table lateral movements executed with the device of the present invention, and FIGS. 23A-C illustrates visual results of table rotation QA, using the device of the present invention.

FIGS. 24A-D illustrate collimator rotation QA, using the device of the present invention.

FIGS. 25A and 25B illustrate the visual results of radiation and light field congruence QA, using the device of the present invention. FIG. 25C illustrates an exemplary embodiment of the user interface showing the acquisition of a 6 MV x-ray QA, as described with respect to FIGS. 25A and 25B. FIG. 25D illustrates another example of a light field and radiation field congruence image acquired using the device of the present invention.

FIG. 26 illustrates a graph obtained using a profile tool in the control program for the device of the present invention.

FIGS. 27A and 27B, 28A and 28B, FIG. 29 and FIGS. 30A and 30B illustrate visual representations of radiation field acquisition and analysis. More particularly, FIGS. 27A and 27B illustrate exemplary plots to show calculation methods for determining photon beam flatness and symmetry using a 1D plot, FIGS. 28A and 28B illustrate visual representations of radiation measurements taken using the device of the present invention, FIG. 29 illustrates a visual representation of a mean radiation image, taken using the device of the present invention, and FIGS. 30A and 30B illustrate exemplary visual representations of energy checks for radiation analysis.

FIGS. 31, 32A-B, and 33A-B illustrate visual representations and analysis of multi-leaf collimator (MLC) QA measurements, taken using a device of the present invention.

FIGS. 33A and 33B illustrate visual representations of measurement and analysis for leaf speed of the MLC.

FIG. 34 illustrates a schematic diagram that the QA device of the present invention can accommodate the measurements of absolute dosimetry with an ionization chamber.

FIG. 35 illustrates an exemplary exposure of the QA device at an angle of collimator rotation in order to assess integrity of collimator rotation.

FIG. 36A illustrates an exemplary exposure of the QA device at a 0° angle of gantry rotation in order to assess integrity of gantry rotation.

FIG. 36B illustrates an exemplary exposure of the QA device at a 30° angle of gantry rotation in order to assess integrity of gantry rotation.

FIG. 37 illustrates the user-interface of the Raven system to measure the rotation integrity of the gantry, collimator and table, according to an embodiment of the present invention.

FIG. 38 illustrates an image view of an irradiated WL bb, according to an embodiment of the present invention.

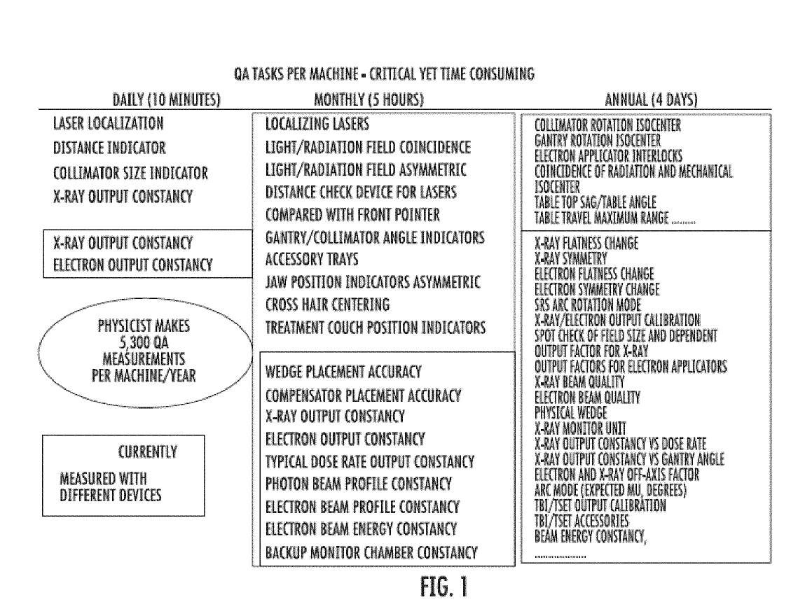

### DETAILED DESCRIPTION OF THE PREFERRED EMBODIMENTS

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

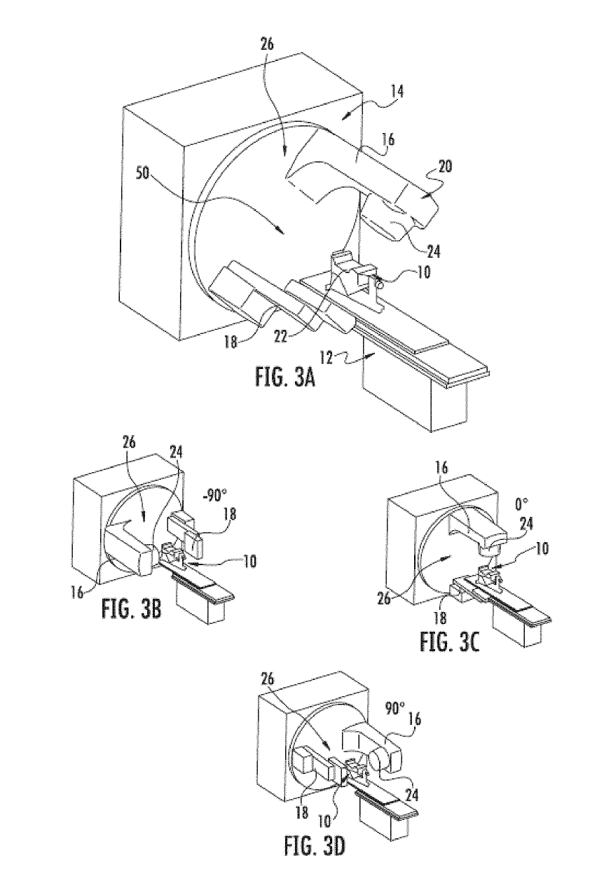

The present invention pertains to a method and apparatus for real-time mechanical and dosimetric quality assurance measurements in radiation therapy. According to an exemplary embodiment as shown in FIGS. 3A-3D, a single unifying device can be employed to perform all mechanical and dosimetric QA tasks, thereby greatly simplifying and unifying all QA tasks. The use of a digital camera ensures that all measurements can be documented for repeat analysis. As such, data can be captured in real-time in one session. In addition, the system can be integrated with the operation of the radiation machines to automate the entire process. The data can be analyzed in real-time or off-line to provide alerts about or to trend the performance of the machine in relationship with the treatment room. Accordingly, the method and apparatus of the present invention unifies measurement of mechanical motion and radiation component of the machine.

FIGS. 3A-3D show the operation principles of the system. In FIGS. 3A-3D, the device 10 is placed on the patient couch 12 of the radiation treatment room. The device 10 is positioned adjacent to a medical accelerator 14 to undergo QA measurements, such that the device 10 is positioned between jaws 16, 18 of the medical accelerator 14 including a multileaf collimeter (MLC) 24 and a gantry 26. When placed on the couch 12, the device 10 can be rotated continuously from approximately +90° to −90°, so as to view the radiation or optical images of the gantry 26 as it rotates from approximately +90° to −90°. The device can also be rotated a full 360°, when it is supported to suspend off the edge of the couch. FIG. 3A illustrates the jaws 16, 18, the gantry 26, and the MLC 24 in an intermediate position between approximately 0° and 90°. FIG. 3B illustrates the jaws 16, 18, the gantry 26, and the MLC 24 in a position at approximately −90°. FIG. 3C illustrates the jaws 16, 18, the gantry 26, and the MLC 24 in a position at approximately 0°, and FIG. 3D illustrates the jaws 16, 18, the gantry 26, and the MLC 24 in a position at approximately 90°.

Also, as illustrated in FIGS. 3A-3D, for radiation QA measurements, including complex intensity modulated treatment, an imaging surface 22 for receiving multiple energy sources will be used. The imaging surface 22 can take any form known to one of skill in the art for receiving the multiple energy sources. More particularly, a phosphor screen or a scintillator sheet can be used to capture the multiple energy sources. The phosphor screen is also used for optical imaging of light fields, optical indicator, and laser paths. The imaging surface 22 can also include markings to show spatial calibration. By placing the device in a position not obstructed by the treatment head 20, the system can be used to capture optically the positions of all lasers and other alignment indicators in the room, as well as the integrity of mechanical motions of the medical accelerator 14 including jaws 16, 18, MLC 24, gantry 26, and couch 12. This is a major improvement since these optical measurements are currently visually noted and recorded as texts in the QA document. The use of a common phosphor screen or scintillator sheet for both radiation and optical imaging is also an important, yet to be reported in the ART, discovery, because it renders un-necessary the replacement of the phosphor screen for optical imaging.

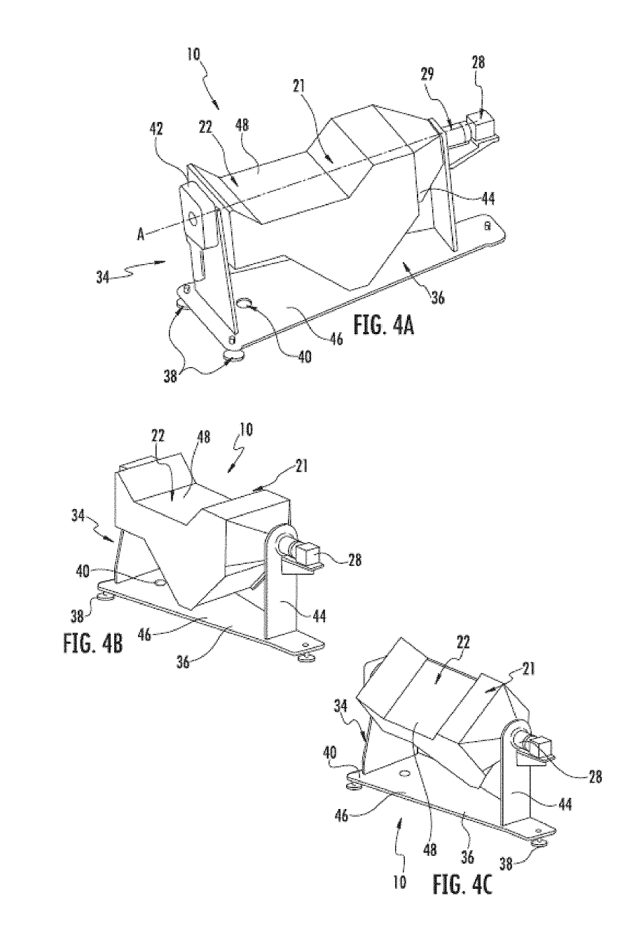

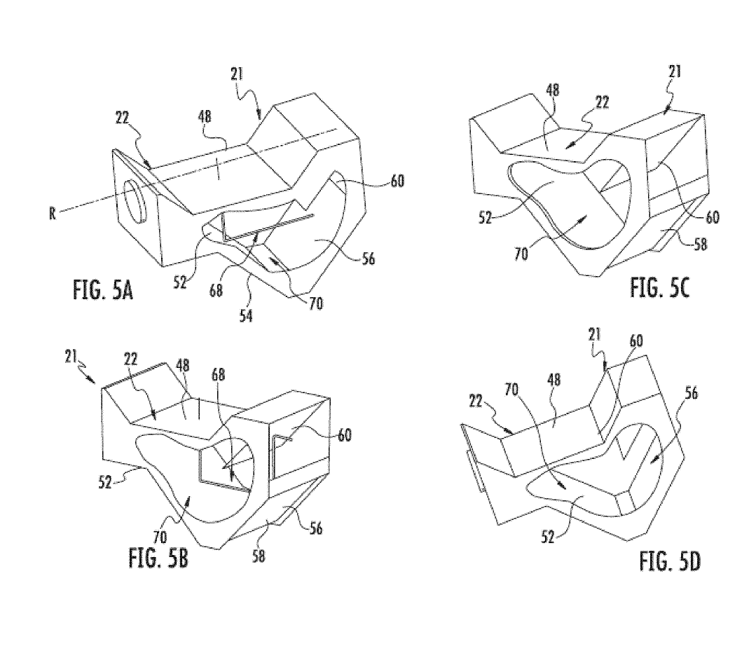

FIGS. 4A-4C illustrate views of the device 10 in further detail according to the features of the present invention. FIG. 4A illustrates a perspective view of a first side of the device 10, while FIG. 4B illustrates a perspective view of a second side of the device 10, both according to the features of the present invention. FIG. 4C illustrates a perspective view of the device rotated to a position of about −45°. As illustrated in FIGS. 4A-4C, the device 10 includes a housing 21 and a camera 28. The camera can take the form of a conventional digital camera or a radiation resistant camera. It should be noted that if a conventional digital camera is used a shield, described further herein, can be used to protect the camera from radiation and to lower the overall cost of the device from a model including a radiation resistant camera. The camera 28 is further configured to measure and record data related to multiple energy sources used in radiation therapy. The camera 28 also includes a lens 29. In order to divert data from the multiple energy sources to the camera 28, at all medical accelerator 14 gantry 26 angles, the device 10 also includes a mirror system 30. The mirror system 30 includes an internal three-mirror arrangement that will be described in further detail with respect to FIGS. 5A-5D. While a three-mirror arrangement is shown here as an exemplary embodiment, any functional mirror system could be used as long as it maintains a stationary camera position while the receptor phosphor screen rotates about the isocenter plane of the accelerator 14 gantry 26.

Further, as illustrated in FIGS. 4A-4C, the device 10 is mounted on a rotary table 34. The rotary table 34 allows for the device 10 to be rotated between approximately −90° and 90° such that the imaging surface 22 and the mirror system 30 can remain aligned with the gantry 26 to record data. The rotary table 34 can also include a base support 36. The base support 36 includes leveling feet 38 and a bubble level 40. The leveling feet 38 and the bubble level 40 allow for the device 10 to be leveled for optimal operation, when placed for a QA analysis of a medical accelerator. The rotary table 34 can also include arms 42, 44, which hold and suspend the device 10, slightly above a top surface 46 of the base support 36. This allows the device 10 to rotate freely about an axis “A” of the rotary table 34. The mechanism for rotation can take the form of any suitable mechanism for rotating the device 10 from approximately −90° to 90°. In another embodiment, the entire base can be designed to rotate with the arms 42, 44 and device 10, such that the measurements can be acquired in full 360° rotation.

FIGS. 5A-5D shows the internal three-mirror arrangement 30 which can be rotated to capture data from different gantry angles. This three-mirror arrangement 30 within housing 21 allows the placement of the image plane 48, i.e., the imaging surface 22, at the plane of the machine isocenter 50 which is the calibration center of all machine parameters. Most importantly, the camera 28 is stationary and positioned to be on the same axis as the imaging plane. As such, the image plane 48 is co-linear with an axis “B” through the longitudinal center of the camera 28. Additionally, the image plane 48 can be rotatable about the axis “B” of the camera 28. It should also be noted that the camera 28 can be kept stationary while the device 10 rotates about the camera 28.

Further, as illustrated in FIGS. 5A-5D, a first mirror 52 is positioned on a first lower wall 54 of the housing 21 of the device 10, across from the camera 28. A second mirror 56 is positioned on a second lower wall 58 of the housing 21of the device 10 and adjacent to the first mirror 52 positioned on the first lower wall 54. The first mirror 52 and the second mirror 56 are separated by an angle. Any suitable angle of separation can be used so long as the data is properly transmitted to the camera 28. A third mirror 60 is positioned in a first plane parallel to a second plane in which the second mirror 56 is disposed. The first plane is separated from the second plane by a distance. The distance can be any distance suitable for transmitting the data to the camera 28. Preferably, all mirrors are set at 45 degree to allow 90 degree reflection. This is not a necessary requirement, as robotics can be used to provide 90 degree reflection, or software can be used to correct imperfect reflection.

Generally, data from the treatment head 20 is transmitted along a trajectory path 68. Trajectory path 68 travels perpendicularly through the imaging surface 22 and reflects off of the first mirror 52 at an approximately 90° angle. Preferably, the angle will be exactly 90°. Imperfect 90° will be corrected by software. The trajectory path 68 then travels across an interior space 70 defined by walls of the device 10 to be reflected off of the second mirror 56 at an approximately 90° angle. The trajectory path 68 then continues vertically to be reflected off of the third mirror 60 at an approximately 90° angle. After being reflected off of the third mirror 60, the trajectory path 68 continues on through the lens of the camera 28 for recording of the data travelling along the path 68. While a specific mirror system is described above, this example is not meant to be limiting. Indeed, the mirror system can be configured such that it has one or more stationary or adjustable-position mirrors or a combination thereof. Additionally, the mirror system can be rotatable in order to capture data from different gantry angles.

Also, as illustrated in FIGS. 5A-5D, as the device rotates, the third-mirror directs all data from the image plane to the camera. The camera can be held stationary to capture any rotated views that can be digitally corrected or the camera can be rotated in its stationary position with the mirror subsystem so that no image correction is needed. The stationary camera offers advantage in the simplicity of set up. A computer can also be included in the system for collecting and analyzing the data. The computer includes a feedback loop for automatic control of the device 10. More particularly, the device 10 can be programmed to move in synchrony with mechanical components of the medical accelerator about the isocenter. Measurements are made in real time. The system can be extended for QA of the kilovoltage imaging system, as well as the positioning of radioactive sources in brachytherapy. Software tools can be implemented to analyze, evaluate and trend the performance of the treatment unit and the integrity of in-room alignment accessories.

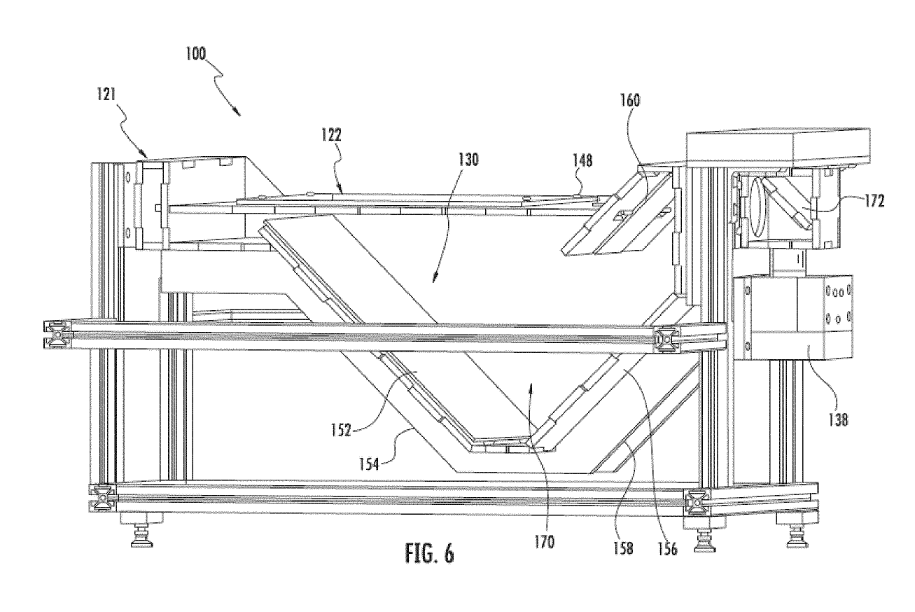

FIG. 6 illustrates a partially sectional view of a QA device according to an embodiment of the present invention. As described above with respect to FIGS. 5A-5D, FIG. 6 illustrates a partially sectional view of the device 100. An embodiment of the internal mirror arrangement 130 is illustrated, within housing 121. The internal mirror arrangement 130 allows the placement of the image plane 148, i.e., the imaging surface 122, at the plane of the machine isocenter.

Further, as illustrated in FIG. 6, a first mirror 152 is positioned on a first lower wall 154 of the housing 121 of the device 100. A second mirror 156 is positioned on a second lower wall 158 of the housing 121of the device 100 and adjacent to the first mirror 152 positioned on the first lower wall 154. The first mirror 152 and the second mirror 156 are separated by an angle. Any suitable angle of separation can be used so long as the data is properly transmitted to the camera. A third mirror 160 is positioned in a first plane parallel to a second plane in which the second mirror 156 is disposed. The first plane is separated from the second plane by a distance. The distance can be any distance suitable for transmitting the data to the camera. The device can also include a fourth mirror 172 positioned, such that it shields the camera from direct radiation from the medical accelerator. The fourth mirror 172 is also positioned at an angle such that the data is transmitted from the third mirror 160 to the camera.

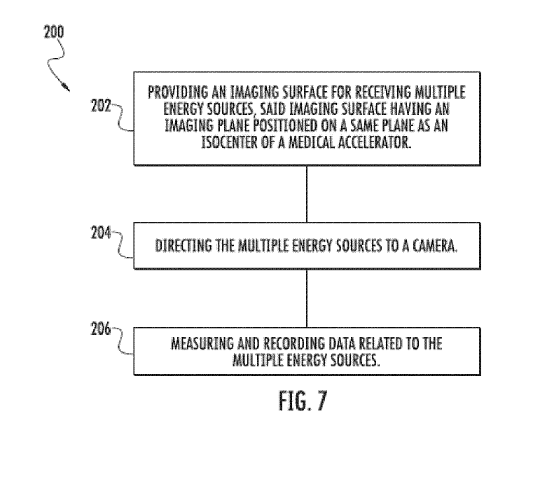

FIG. 7 illustrates a diagram of a method in accordance with the features of the invention. The method 200 is directed generally to real-time mechanical and dosimetric quality assurance measurements in radiation therapy. The method 200 includes a step 202 of providing an imaging surface for receiving multiple energy sources. More particularly, the imaging surface has an imaging plane positioned on a same plane as an isocenter of a medical accelerator. Step 204 includes directing the multiple energy sources to a camera. Additionally, step 206 can include measuring and recording the data related to the multiple energy sources.

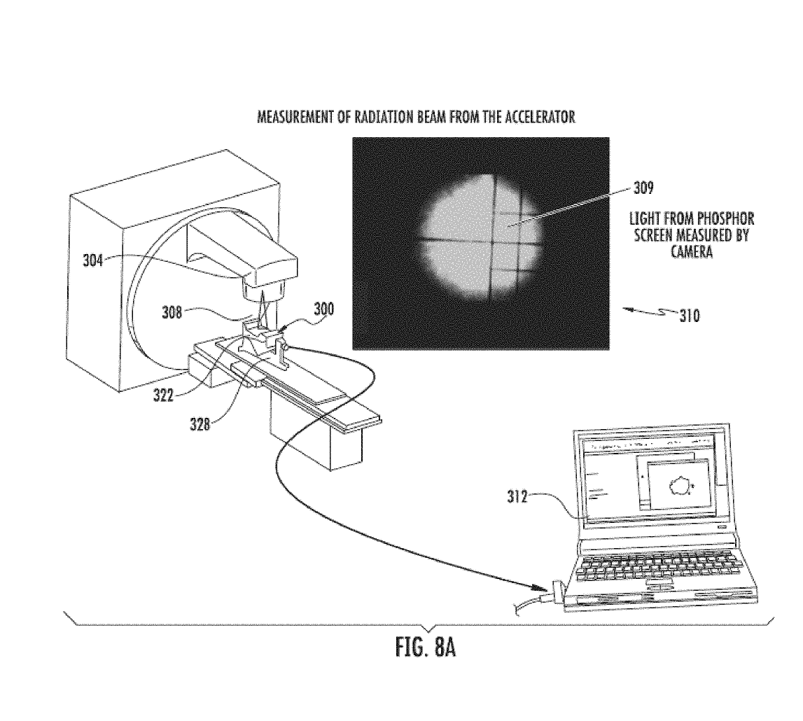

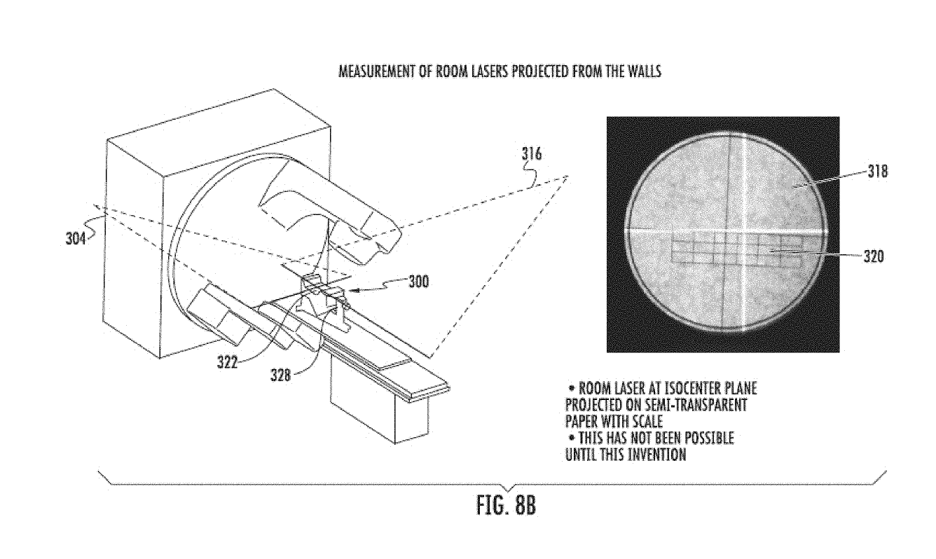

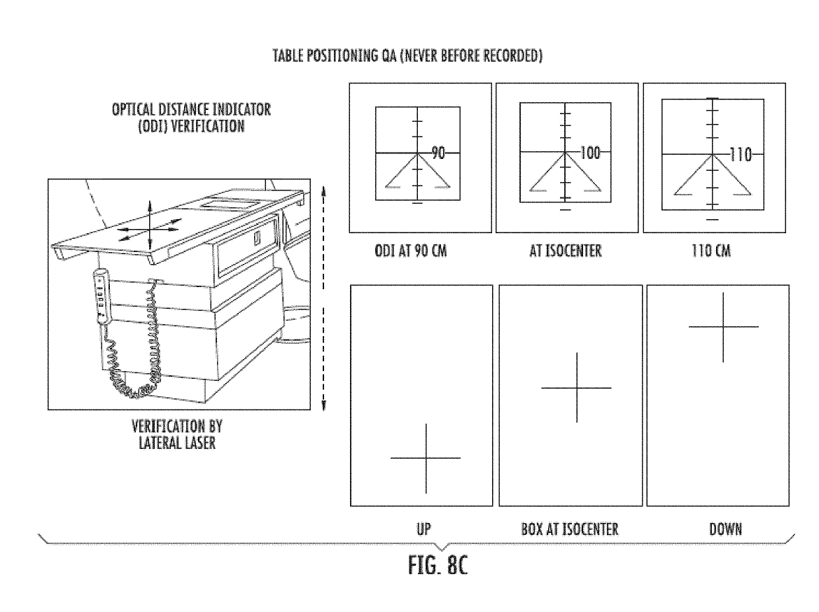

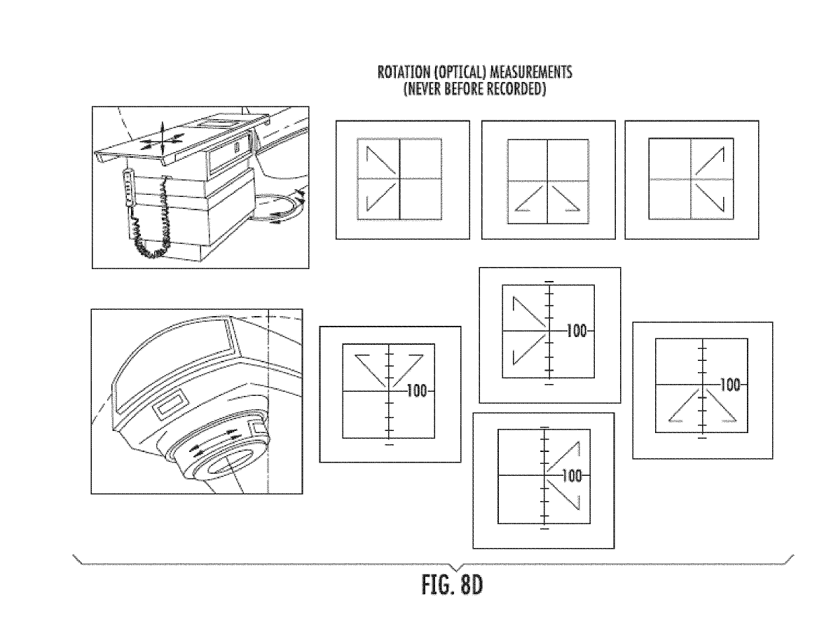

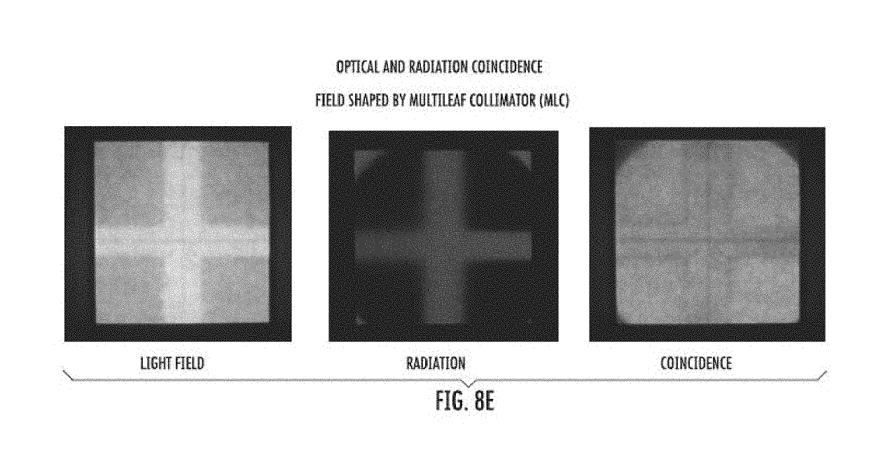

FIGS. 8A and 8B further illustrate methods of assessment of two of the many QA criteria to be performed on a medical accelerator, in accordance with the features of the invention. As shown in FIGS. 8A and 8B, multiple energy sources can be directed at the device 300, and can thus be recorded by the device 300. In FIG. 8A, a radiation beam 308 from the medical accelerator 304 is directed at the device 300. The radiation beam 308 is directed at the device 300, such that it travels through the imaging surface 322, in this case a phosphor screen. The light 309 from the phosphor screen can then be measured by the camera 328, as shown in sample image 310. The camera 328, can then transmit the data to computer 312 for recording and analysis, either through a wired or wireless connection. In FIG. 8B, room lasers 314, 316 at isocenter plane can be directed at an imaging surface 322 of the device 300. The phosphor screen can also be used as the image receptor 318 at the imaging plane 322 with an overlaying scale 320 or a digitally encoded scale. The projection of the room lasers 314, 316 is measured by the camera 328, and the data can then be transmitted to a computer (not pictured) for recording and analysis.

FIG. 8C illustrates a method of performing table positioning QA, according to an embodiment of the present invention. Previously, table positioning QA was measured visually. Optical Distance Indicator (ODI) verification is used, which is a verification of a lateral laser. The laser, which mimics the source of radiation, is shined onto the device of the present invention, which is disposed on the patient table and rotated 90° to view the lateral room laser. The correctness of the positioning of the laser was previously assessed visually, and no recording of the positioning could be made. Using the device of the present invention, the ODI can be recorded and assessed. The recording can also be saved for any later review, or verification of the proper function of the medical accelerator. As illustrated in FIG. 8C, the ODI is recorded at 90 cm, isocenter (or 100 cm), and 110 cm. FIG. 8C also illustrated the table in an up or lifted position, at isocenter, and in a down or lowered position.

FIG. 8D illustrates a method of performing rotation or optical QA, according to an embodiment of the present invention. This method measures the accuracy of the rotation of the collimator, and before the device of the present invention, this QA measurement was not recorded. To check collimator rotation, a technician directs a light source that mimics the radiation beam onto the device of the present invention, which is disposed on the patient couch. The technician rotates the collimator to observe that the light moves correctly about the isocenter of the medical accelerator. As illustrated in FIG. 8D, the rotation of the collimator is measured about the isocenter (100 cm). Rotation of the collimator is measured at rotations of 90, 180, 270, and 360 degrees. Using the device of the present invention, the position of the light source can be recorded, as illustrated in FIG. 8D.

FIG. 8E illustrates a method of performing optical and radiation coincidence of a field shaped by the multi-leaf collimator. As illustrated in FIG. 8E, the device of the present invention can be used to record not only a light field shaped by the multi-leaf collimator, but also a radiation field shaped by the multi-leaf collimator. Further as illustrated in FIG. 8E the coincidence of the light field and the radiation field can be superimposed with the radiation image in order to ensure accuracy of the radiation field. The recorded images can be kept to show proper function of the medical accelerator. Additionally, like the methods of measurement of QA discussed with respect to FIGS. 8C and 8D, the method of performing optical and radiation coincidence has not previously been recorded.

Additionally, software can be incorporated into a system of operating the present invention. Incorporating software would allow the present invention to be further automated, saving even more time for a medical physicist performing QA on a medical accelerator. The device of the present invention can include a microprocessor, computing device, or other means of providing computer control to the device known to or conceivable by one of skill in the art. Alternately, the software can be loaded onto a separate computing device, server, or remote server and can communicate wirelessly or with a wired connector to a control device housed in the device of the present invention. Such a set-up would allow for multiple QA devices to be controlled by one separate computing device, server, or remote server. Any other software control set-up known to or conceivable by one of skill in the art could also be used. The device can also include robotic control to translate movement commands from the computer program into movement of the device. Additionally, the operation of the device can be integrated with the operation of the medical accelerator

For instance, as far as mechanical features, the software can be used to control or measure at least the colinearity and convergence of the lasers, table movement and optical distance indicator, as well as collimator movement. With respect to radiation features, the software can be used to control or measure at least light and radiation field congruence, radiation profile constancy, and energy constancy. The light and radiation field congruence can be controlled at least with respect to the x-ray beams, flatness and symmetry, and conformity index. The radiation profile constancy can be controlled at least with respect to electron beams, and the energy constancy can be controlled at least with respect to x-ray and electron beams.

Another software tool can be configured for setting the camera. In performing a QA analysis, the technician can use the software to set the camera, including but not limited to setting the time of integration and a number of frames to be acquired. The settings can be stored as a file in order to streamline QA testing further. For instance, settings for optical related QA testing can be stored as a file, while settings for radiation related QA testing can be stored as a separate file. When the technician is ready to engage in either QA testing protocol, he can use these stored software files to configure the camera to the appropriate settings for the type of testing. These settings files can be dated, so that the technician knows which file is most recent. The file can also be called up on the computing device in order to allow the technician to view and verify the settings when the file is called up for view on a screen of the computing device.

The software can also be configured to control image acquisition. However, if desired the technician can still manually acquire images using the camera. In an exemplary image acquisition, the technician can select the setting file, described above, acquire an image, and name that image to save it to a hard drive or networked drive of the computing device. After the image is named and saved, it can remain on the screen, such that the technician can complete various analysis tasks using the software.

For simple, efficient and effective image analysis purposes, the software can be configured to display a hard coded digital image scale, as illustrated in FIG. 9. The digital image scale can be superimposed over the acquired image, and the technician can also have the option of turning the digital image scale.

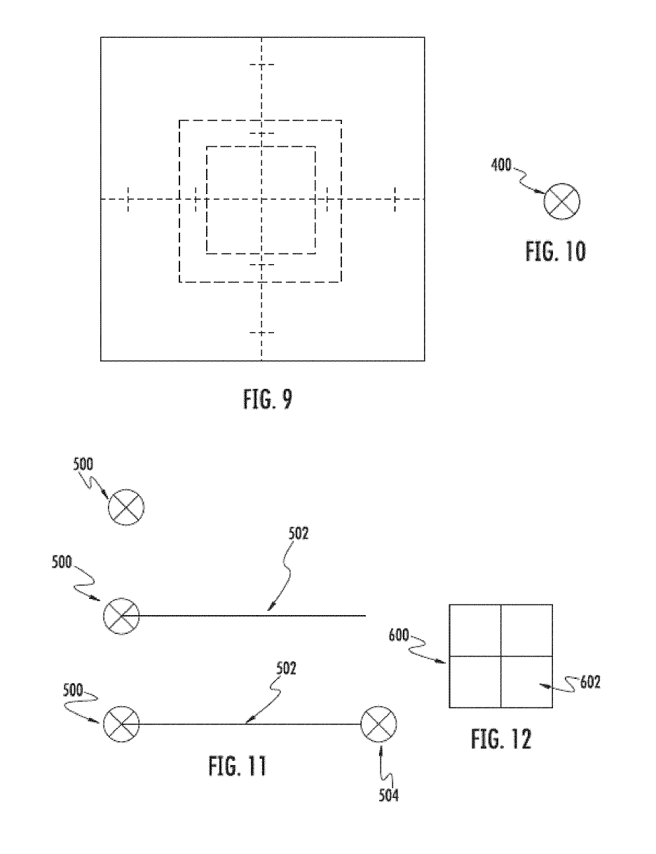

FIG. 10 illustrates an analysis tool, provided by the software control program for the device of the present invention. As illustrated in FIG. 10 the analysis tool can drop a circle 400 having a diameter of 10 pixels, at a point of interest in the image acquired using the device of the present invention. The pixel location of interest can also be recorded using the software control program, per requirements for various QA tasks. It should be noted that the diameter of the circle can be any number of pixels found suitable to one of skill in the art.

FIG. 11 illustrates a localization tool, provided by the software control program for the device of the present invention. As illustrated in FIG. 11 the localization tool can drop a first circle 500 having a diameter of 10 pixels. The localization tool then allows the technician to drag a line 502 to a second point of interest, where a second circle 504 can be dropped. In order to adjust the placement of the analysis tool, the line 502 can be stretched or shrunk. The software program can then be configured to determine and display the number of pixels defined by the line. If scale is available, the software program can be used to show the length of line 502 in mm.

FIG. 12 illustrates a region of interest tool, provided by the software program for the device of the present invention. The region of interest tool 600 can take the form of a square with crosshairs 602. Preferably, the region of interest tool 600 is 20 or more pixels in diameter depending on the camera resolution, to be roughly 5 mm. However, the region of interest tool can be configured to take any other suitable size known to or conceivable by one of skill in the art. The region of interest tool 600 can be a drag and drop tool, such that the technician can move it around to various regions of the image. Related tools could include an option to select pixels at the intersection of the crosshairs. Image statistics could also be calculated, such as mean pixel intensity and standard deviation. Additionally, the software can be configured to store user name and date on the image and also any specific information regarding pixel coordinates and statistics.

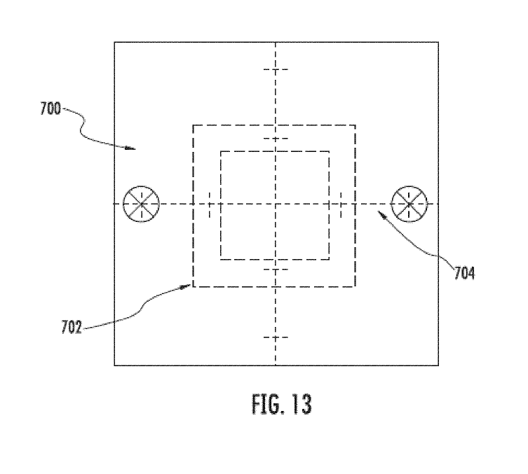

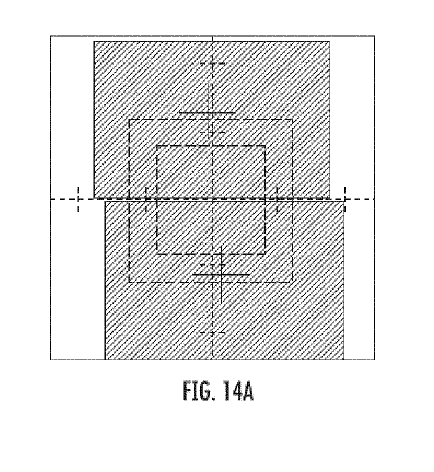

FIG. 13 illustrates an exemplary screen for defining scale in an acquired image taken using the device of the present invention. In the example, the technician acquires an optimal image 700 applies the digital image scale 702 described with respect to FIG. 9, and sets up a digital scale image. The technician can then call up a ruler tool to draw out a line 704. When the rule tool is called up, a dialog box can open, requesting a distance of the line in mm. The technician can confirm the distance, and the program calculates the scale in mm/pixel. The program can also be used to date and store the scale image. The scale created using this process is considered the default until it is remeasured and updated.

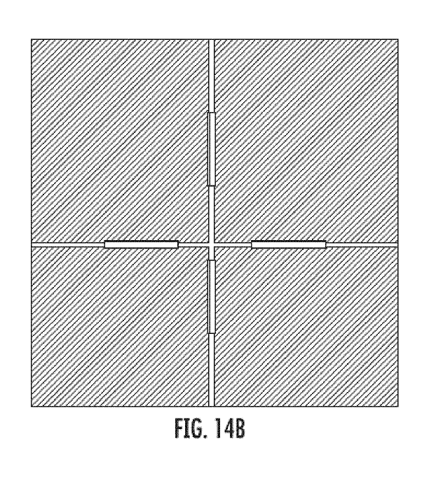

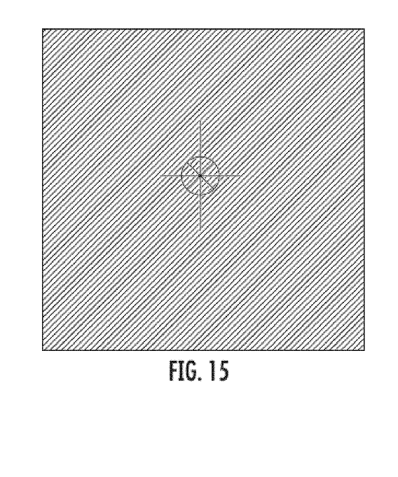

FIG. 14A illustrates an example of a program feature to superimpose images taken with the device of the present invention. A “superimpose” function key retrieves n number of images and superimposes the images on one another, as illustrated in FIG. 14A. More particularly, FIG. 14A illustrates an example with two laser images superimposed with a ruler image map. FIG. 14B illustrates an exemplary recording of laser QA to analyze the co-linearity of the laser, using the device of the present invention. If the lines of the laser in the image form a straight line, the medical accelerator receives a pass, but if they do not the medical accelerator receives a fail.

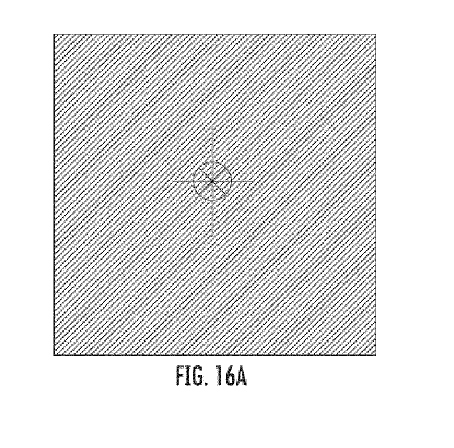

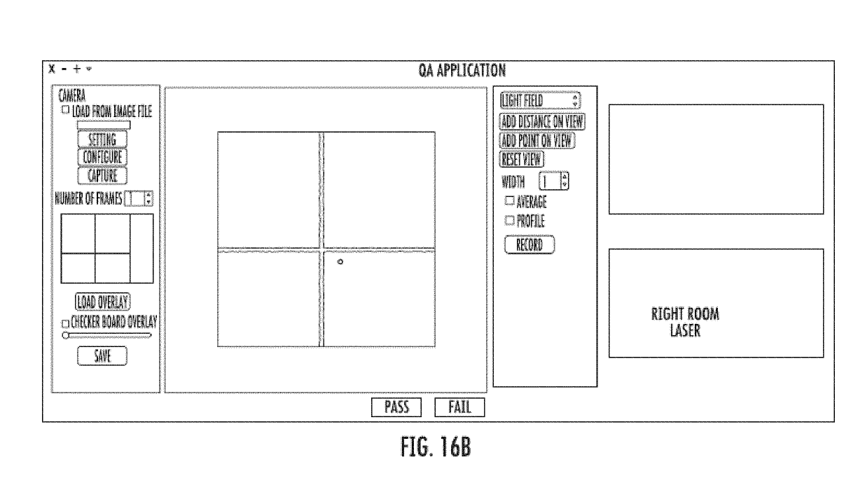

FIGS. 15-19 illustrate exemplary recordings of laser QA tasks executed with the device of the present invention. For instance, FIGS. 15 and 16A illustrate acquiring left and right lateral laser QA with the device of the present invention. The technician can select Laser QA tasks from a drop down menu. The Laser QA tasks list then includes options to perform QA for the left side of the room, the right side of the room, the ceiling of the room, and the back wall of the room. For left and right lateral QA, the program instructs the technican to acquire an image of a laser at the isocenter. The technician then localizes the laser crosshair using the pixel analysis tool described with respect to FIG. 10. The technician can then name and store the image. The technician is then prompted to acquire a second laser image after moving the treatment couch approximately 10 cm towards the laser. The technician then acquires a second image of the laser, and localizes the laser crosshair using the pixel analysis tool. This image is also named and stored. FIG. 16B illustrates an exemplary embodiment of the user interface showing the acquisition of a right room laser QA, as described with respect to FIG. 16A.

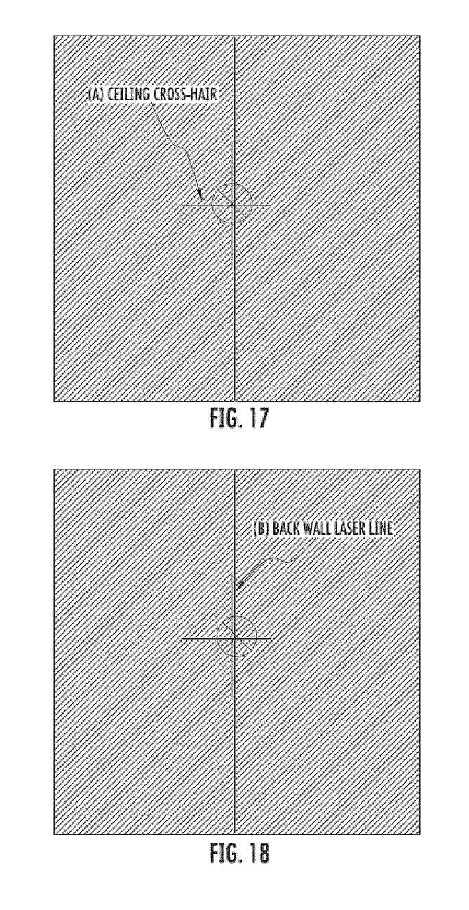

FIGS. 17 and 18 illustrate obtaining ceiling and back wall lateral laser QA, respectively, using the device of the present invention. In these examples, the technician acquires an image of the ceiling and the back wall lasers at isocenter. For FIG. 17, the technician localizes the laser cross-hairs and names and stores the ceiling laser image. The technician can then localize the intersection of the back wall and ceiling lasers, as illustrated in FIG. 18. The technician can then acquire another image after moving the treatment couch up toward the ceiling by 10 cm. The localization, naming, and storing procedures can be repeated for this image.

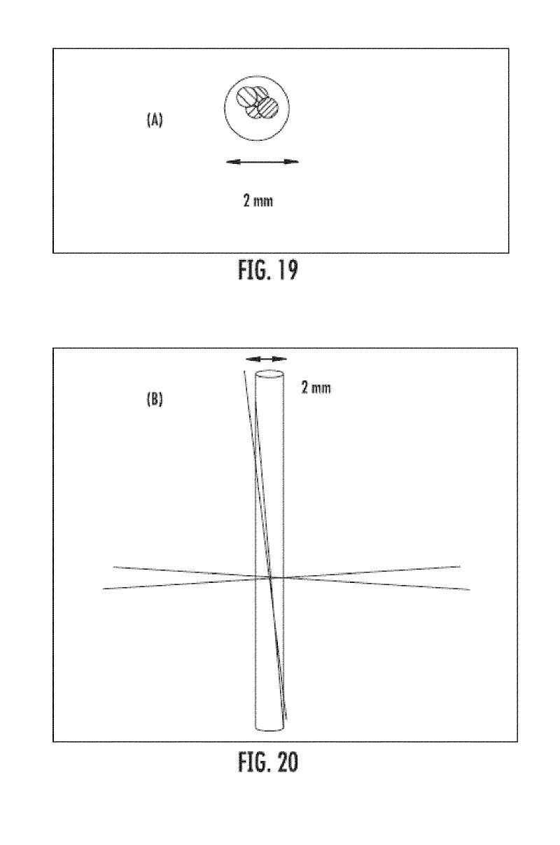

FIGS. 19 and 20 illustrate laser alignment analysis using the device of the present invention. The technician can request that the program perform laser analysis using the program menu. The program with combine isocenter locations for all laser crosshairs in a 2D or 3D view. The program will denote the lasers as a pass, if they are contained within a 2 mm diameter, as illustrated in FIG. 19. The program then derives co-linearity for each laser direction from the paired laser images, as illustrated in FIG. 20. The program will denote the lasers as a pass if the line is contained within a 2 mm diameter cylinder orthogonal to the isocenter over 20 cm.

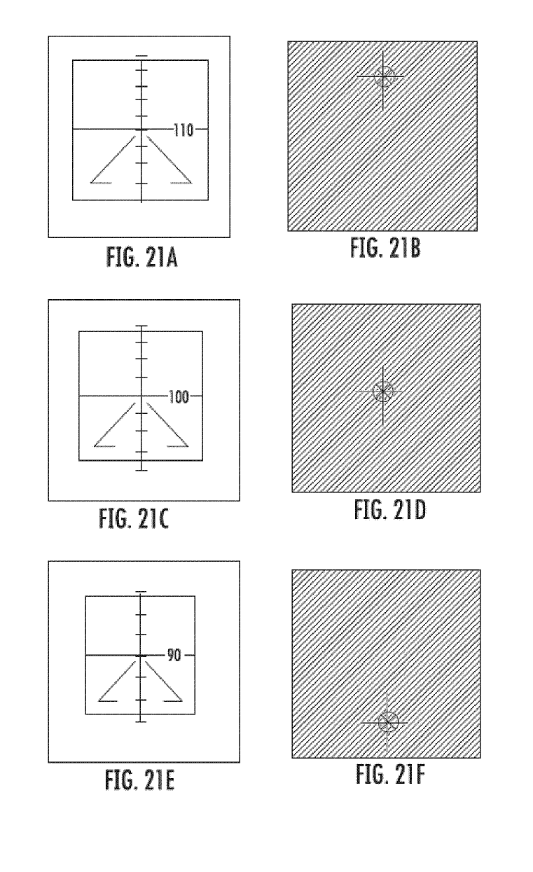

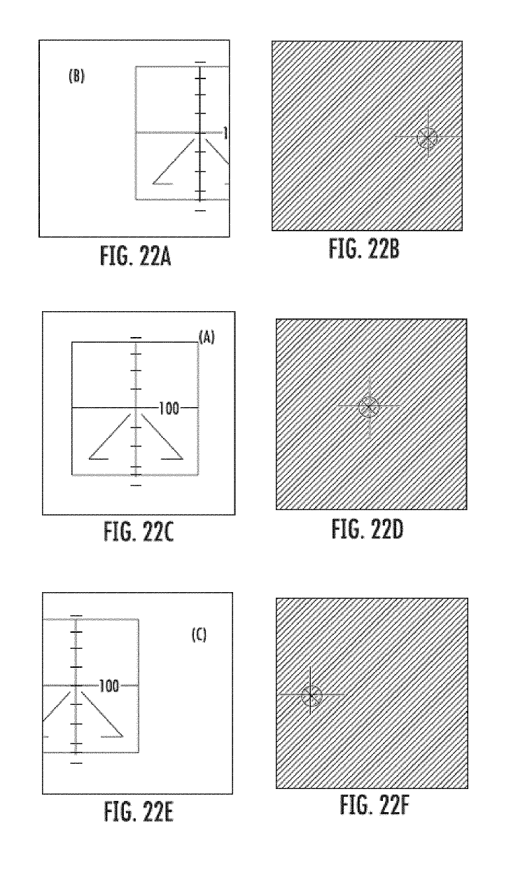

FIGS. 21A-F, 22A-F, and 23A-C illustrate exemplary recordings of table movement and optical distance indicator (ODI) QA tasks executed with the device of the present invention. FIGS. 21A-F relate to QA for table vertical movements captured using the device of the present invention. The program can be used to set the QA box to horizontal to acquire an ODI image at 110 cm SSD, as illustrated in FIG. 21A. The box can also be set manually and the images processed using the program. The QA box is then rotated vertical 90° to acquire a left or right laser image at 110 cm SSD as illustrated in FIG. 21B. This procedure is repeated for 100 cm SSD as illustrated in FIGS. 21C and 21D and 90 cm as illustrated in FIGS. 21E and 21F. The images can all be named and stored. The superimpose function, described above with respect to FIG. 14, can be used to measure vertical distance of the localized laser against the digital scale. The ruler tool can also be used. The program will denote this QA as a pass if the distance delta is within 1 mm.

FIGS. 22A-F relate to QA for table lateral movements executed with the device of the present invention. The program can be used to set the QA box horizontal to acquire the optical field image at 100 cm SSD, as illustrated in FIG. 22A. The box can also be set manually and the images processed using the program. The technician also needs to acquire an image of the ceiling laser as illustrated in FIG. 22B. The procedures are repeated for a table move 10 cm to the left of center, as illustrated in FIGS. 22C and 22D and 10 cm to the right of center, as illustrated in FIGS. 22E and 22F. The superimpose function, described above with respect to FIG. 14, can be used to measure vertical distance of the localized laser against the digital scale. The ruler tool can also be used. The program will denote this QA as a pass if the distance delta is within 1 mm.

FIGS. 23A-C illustrates visual results of table rotation QA, using the device of the present invention. The QA box is set horizontal at the isocenter of the medical accelerator, either manually, or using the program. Optical images of the light field are acquired at table rotations of 180°, 90°, and 270°, as illustrated in FIGS. 23A-C, respectively, or any arbitrary but known angles. The crosshairs are localized in each image and the image is tagged and stored. The images can be superimposed using the feature discussed with respect to FIG. 14. The localized crosshairs must be within a circle of a 2 mm diameter in order for the system to pass this QA analysis.

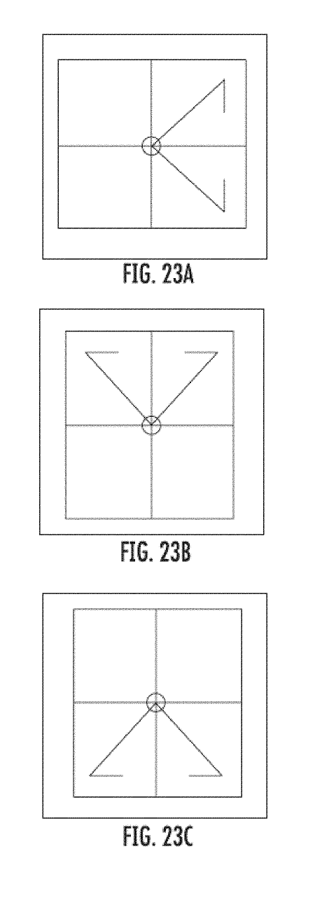

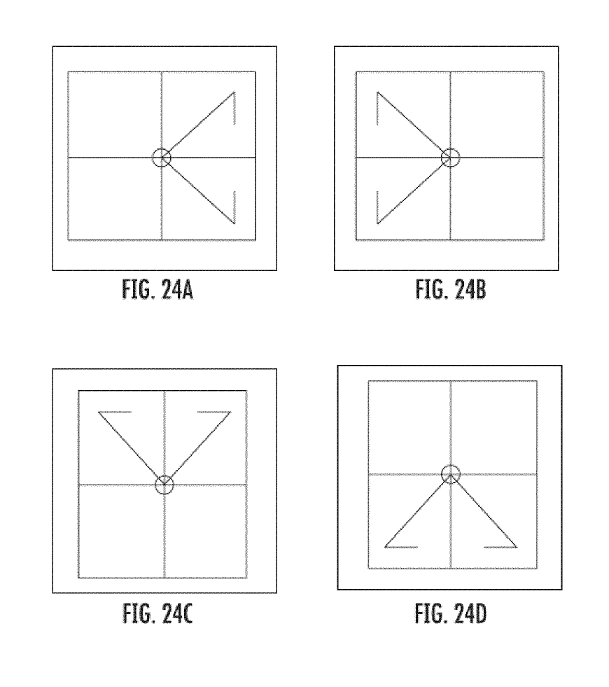

As illustrated in FIGS. 24A-D collimator rotation QA is executed in much the same manner as table rotation QA, using the device of the present invention. The QA box is set horizontal at the isocenter of the medical accelerator, either manually, or using the program. Optical images of the light field are acquired at collimator rotations of 180°, 0°, 90°, and 270°, as illustrated in FIGS. 24A-D, respectively, or any arbitrary but known angles. The crosshairs are localized in each image and the image is tagged and stored. The images can be superimposed using the feature discussed with respect to FIG. 14. The localized crosshairs must be within a circle of a 2 mm diameter in order for the system to pass this QA analysis.

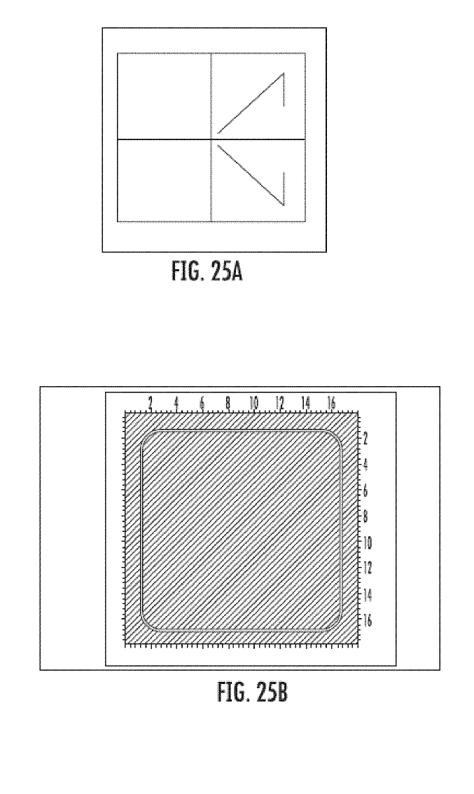

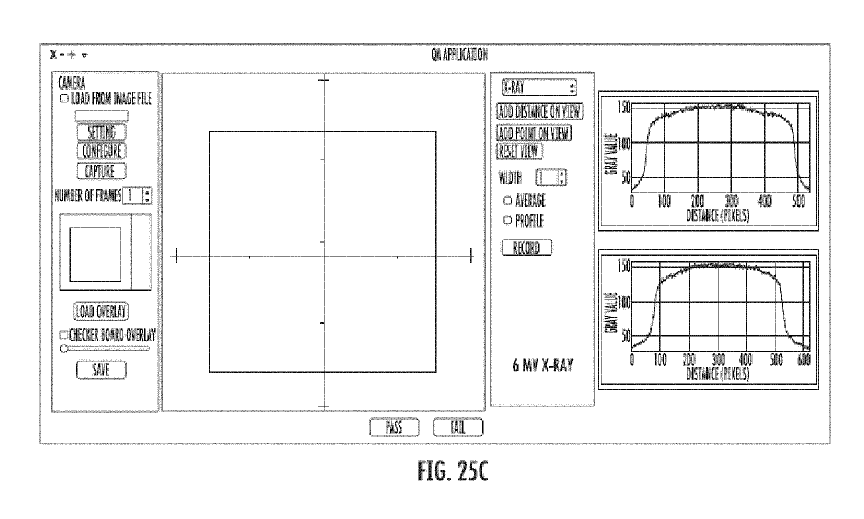

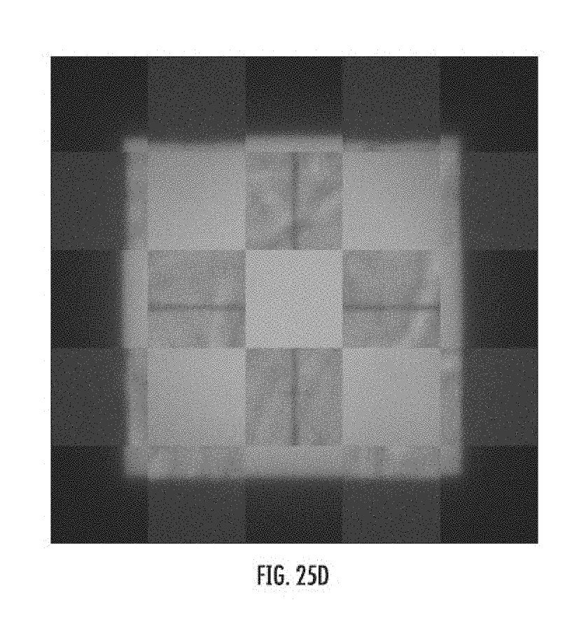

FIGS. 25A-D, 26, 27A and 27B, and 28A and 28B illustrate the visual results of radiation and light field congruence QA, using the device of the present invention. For general radiation field acquisition, which can be executed manually or using the device control program, the radiation camera setting is selected to acquire dark current image with the camera shut for 15 sec or 3×15 sec. Radiation exposure is acquired for the same exposure setting and camera-lens non-uniformity correction is applied if applicable. A mean dark current image can be subtracted from the “corrected” mean radiation image.

Particularly, as illustrated in FIGS. 25A-25D a radiation/light field congruence tool can be used to produce images similar to the examples shown in FIGS. 25A and 25B. The optical camera setting is selected either manually or using the device control program, in order to acquire a light field at preset dimensions, such as 20 cm×20 cm. A previously stored image can also be used. The program can then be used to detect a light field boundary. The radiation camera setting is used to acquire and process dark current and a radiation field for the same dimensions. Radiation field boundary can then be detected at an intensity level of 50%. The light field boundary and the radiation field boundary can then be overlaid and the image named and stored. FIG. 25C illustrates an exemplary embodiment of the user interface showing the acquisition of a 6 MV x-ray QA, as described with respect to FIGS. 25A and 25B. FIG. 25D illustrates another example of a light field and radiation field congruence image acquired using the device of the present invention.

FIG. 26 illustrates a graph obtained using a profile tool in the control program for the device of the present invention. In order to use the profile tool a radiation image must be obtained. The program can then be asked to display a 1D plot of x- or y-profiles, such as the graph of FIG. 26. The light field boundaries 600 can also be displayed on the graph. The graph can be used to calculate flatness, symmetry, and uniformity index according to formulae. The graph and results can be stored for future reference.

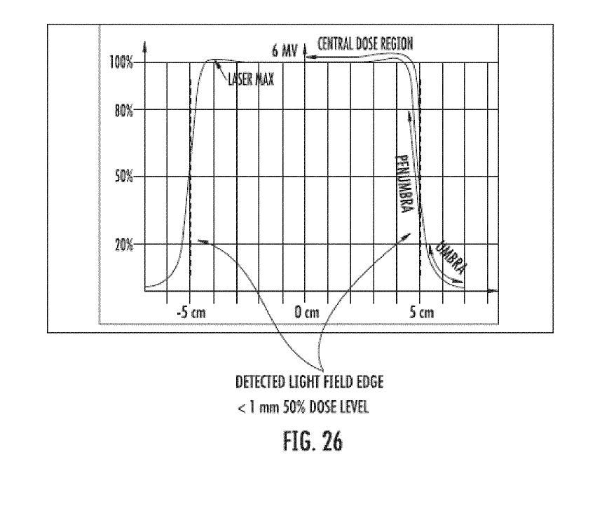

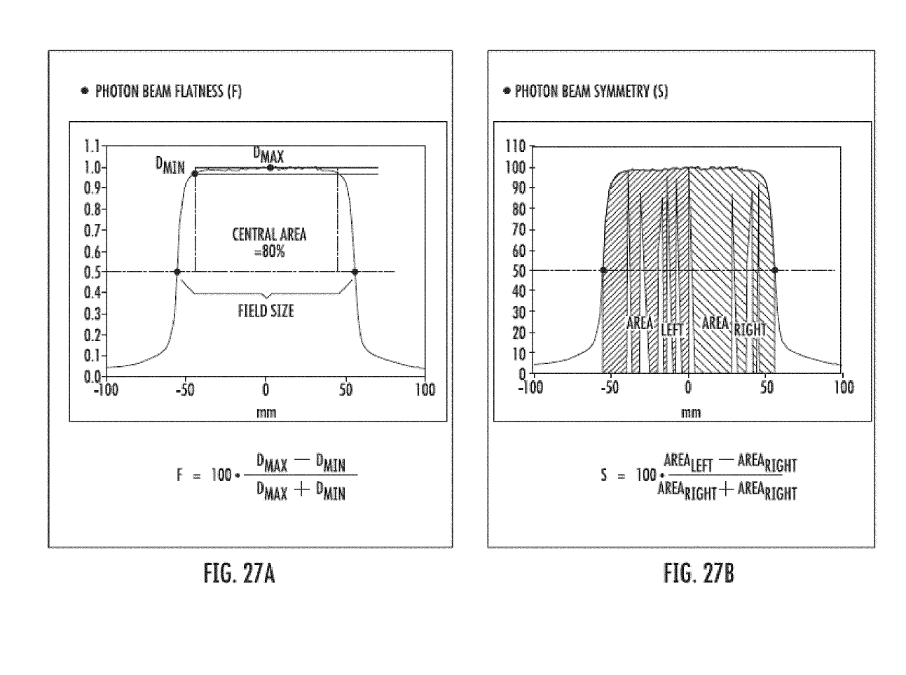

FIGS. 27A and 27B illustrate exemplary plots to show calculation methods for determining photon beam flatness and symmetry using a 1D plot, like the one illustrated in FIG. 26. The photon beam flatness (F) calculation is illustrated in FIG. 27A, where F=100*(Dₘₐₓ−Dₘᵢₙ)/Dₘₐₓ+Dₘᵢₙ). The symmetry (S) calculation is illustrated in FIG. 27B, where S=100*(area_{left}−area_{night})/(area_{left}+area_{night}). The graph and results can be stored for future reference.

FIGS. 28A and 28B illustrate visual representations of radiation measurements taken using the device of the present invention. FIG. 28A illustrates a visual representation of the radiation field, and FIG. 28B illustrates a calculation of the uniformity index (UI), where UHarea=(area_{90%})/(area_{50%}). The graph and results can be stored for future reference.

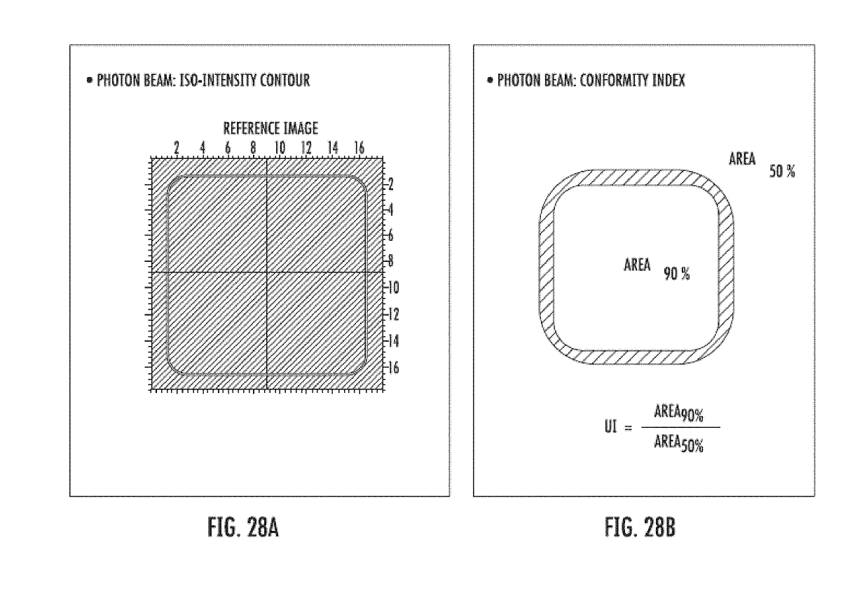

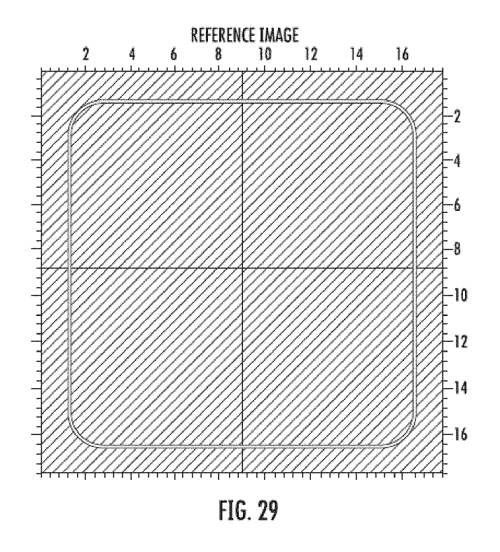

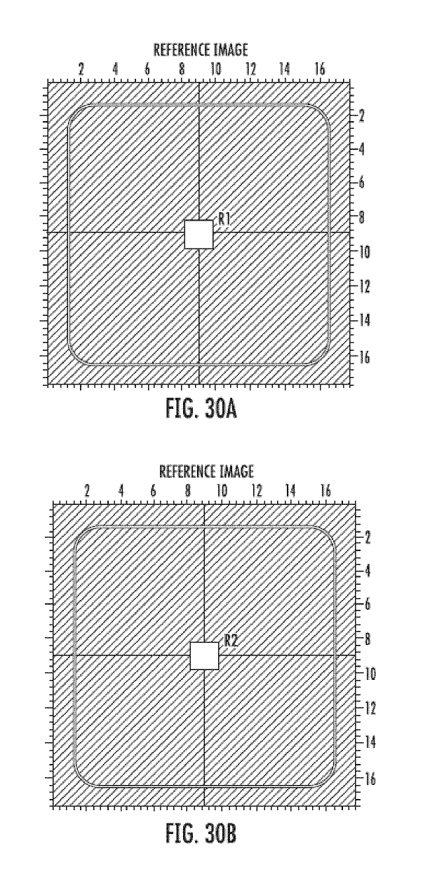

FIG. 29 and FIGS. 30A and 30B illustrate visual representations of radiation field acquisition and analysis. It should be noted that when radiation fields are imaged using the device of the present system, a simultaneous film measurement can also be taken. The ratio of the film measurement to the measurement from the camera of the device of the present invention can then be used to provide a one-time correction map that can be used for every test measurement. More particularly, FIG. 29 illustrates a visual representation of a mean radiation image, taken using the device of the present invention. The device can measure any radiation field such as 4, 6, 8, 10, 15, or 18 MV x-rays or 6, 8, 1−, 12, 15, and 18 MeV electron beams. Dark current and radiation images are acquired according to the protocols described above. The images are then processed by the program to create a mean radiation image. The image can be named and stored for further reference or additional calculations such as flatness, symmetry and uniformity index, all described previously.

FIGS. 30A and 30B illustrate exemplary visual representations of energy checks for radiation analysis. To perform the energy checks the technician acquires a pair of appropriate mean radiation images taken under different thicknesses of a material such as plastic or simulated solid water. A region of interest is placed at a center of the radiation image such as Reading 1 (R1) and Reading 2 (R2) in FIGS. 30A and 30B. R1=the average count for the region of interest for the first image, and R2=the average count for the region of interest for the second image. The energy constant ratio=R1/R2. These results can also be named and stored for future use or reference.

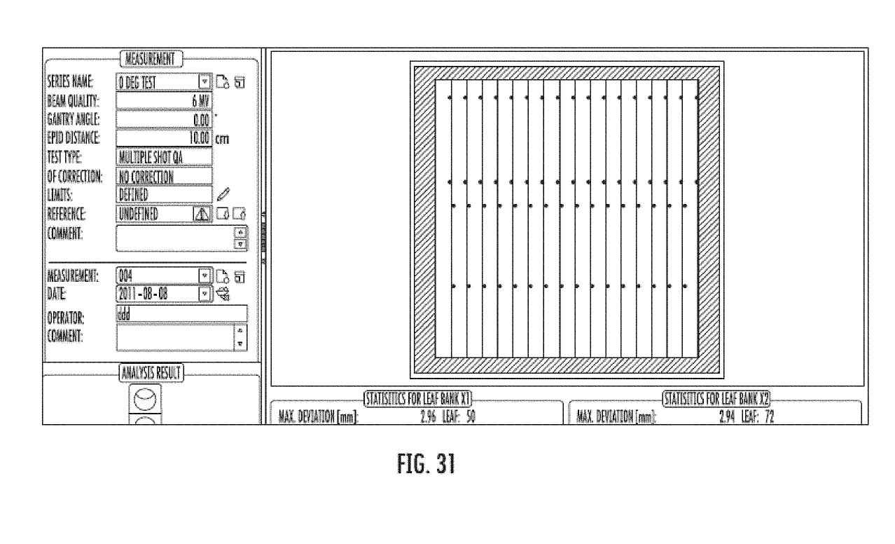

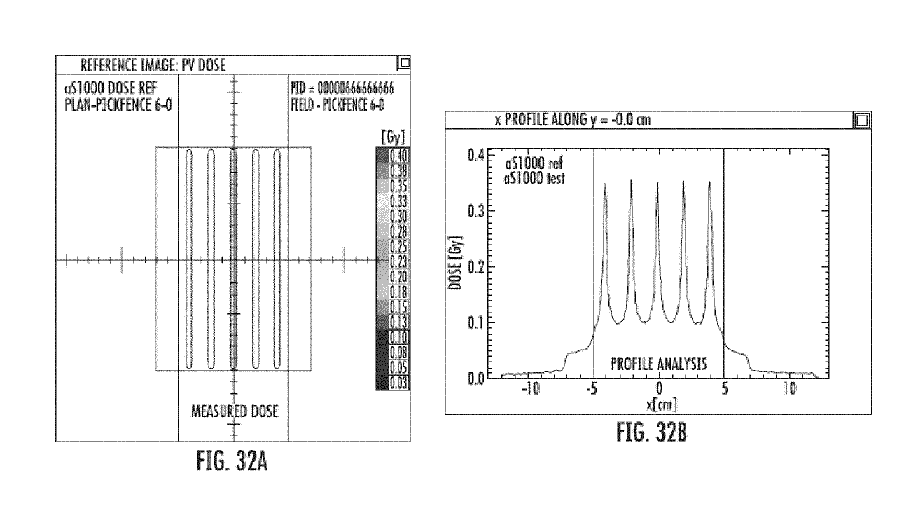

FIGS. 31, 32A-B, and 33A-B illustrate visual representations and analysis of multi-leaf collimator (MLC) QA measurements, taken using a device of the present invention. These analyses ensure MLC positioning accuracy using garden fence delivery, and leaf speed accuracy. With respect to FIGS. 31 and 32A-B, which illustrate visual representation of garden fence analyses, a garden fence image is acquired by setting an integration camera time to 30 sec or more. Imaging begins and the technician drives a slit beam, formed by X1, X2 MLC in the step and shoot mode to deliver radiation to known positions in the field. After delivery and imaging is stopped the positional accuracy of the leaf gap can be analyzed, as in FIGS. 31, and 32A-B.

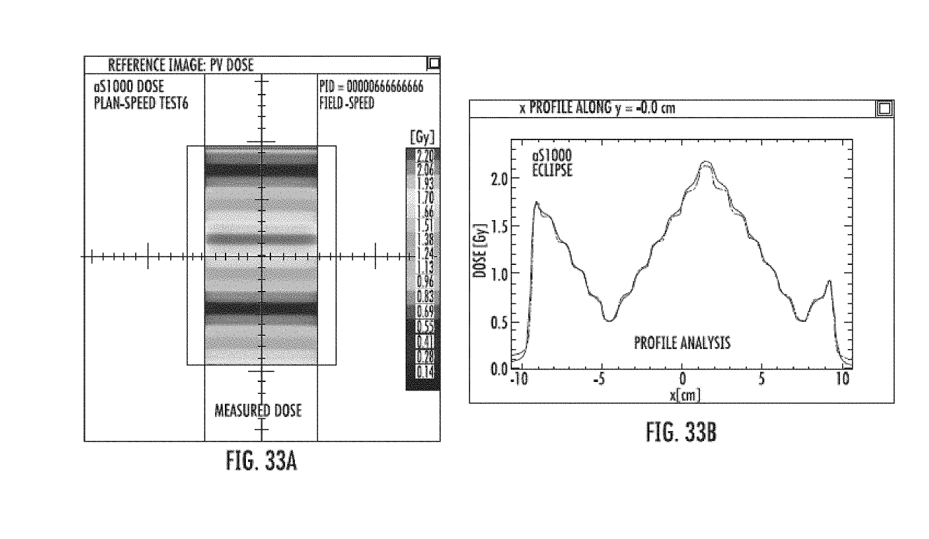

FIGS. 33A and 33B illustrate visual representations of measurement and analysis for leaf speed of the MLC. An image is acquired by setting an integration camera time to 30 sec or more. Imaging begins and the technician drives each leaf pair with different speeds across the field to deliver different doses for each leaf pair. After delivery and imaging is stopped the positional accuracy of the dose profile can be analyzed, as in FIGS. 33A-B.

The above described software tools for automating the QA process and analyzing the images taken with the device of the present invention are exemplary and are not to be considered limiting. Many other software tools could be developed to further automate the process and provide for analysis of the images. The control program for the present invention can also be configured to produce a report outlining all of the QA measurements executed and the results of those measurements.

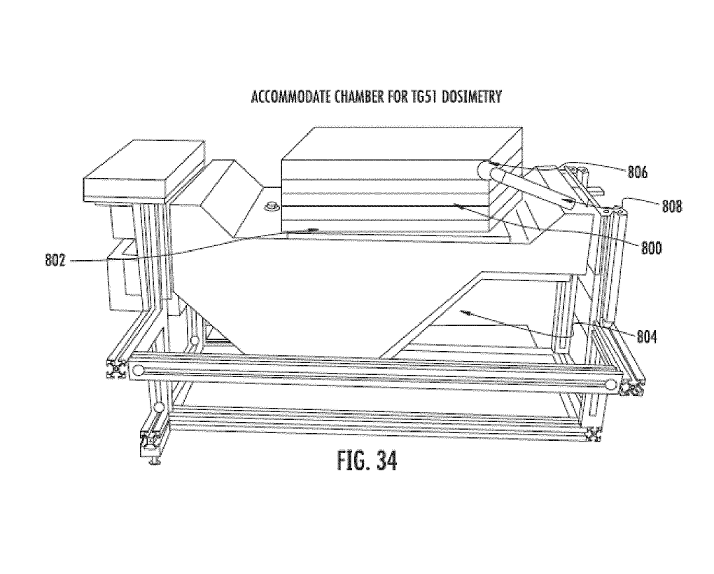

FIG. 34 illustrates an ion chamber for use with a device for QA of a medical accelerator, according to an embodiment of the present invention. The ion chamber 800 can take the form of sheets of plastic water 802 positioned on a surface of the device 804. The sheets of plastic water 802 can be up to 10 cm thick. Additionally, one sheet can include a receptor 806 for an ion chamber 808. (There are no numbering in the Figure) The receptor 808 can be drilled at a 45° angle to a x-y axis of the chamber 800. This configuration allows for TG51 calibration to be performed.

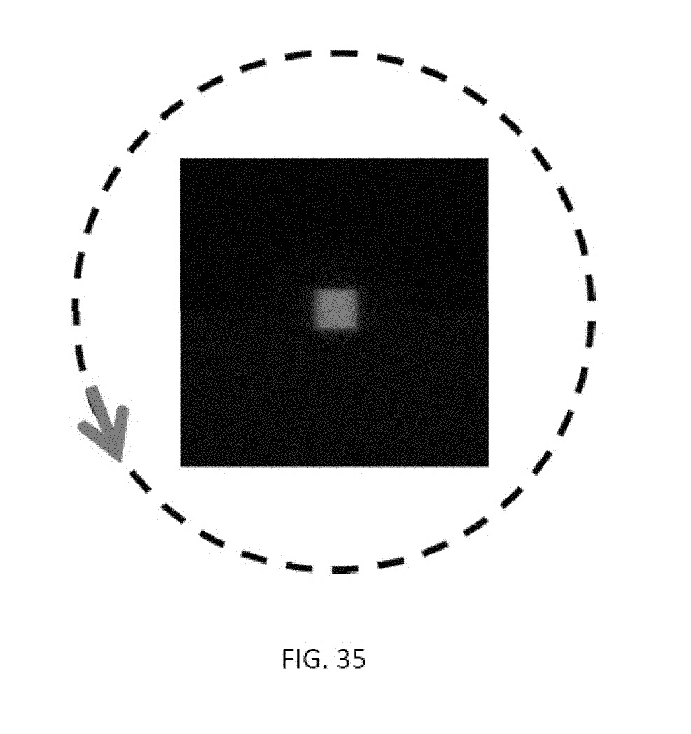

A method in accordance with the present invention includes using a QA device of the present invention, described above, to assess the integrity of collimator rotation. The method includes setting the QA device flat at the isocenter of the collimator and/or the couch. Preferably, the QA device is positioned such that the QA device is hanging off an end of the couch. A small square field of approximately 2 cm×2 cm, or any other suitable field size known to or conceivable by one of skill in the art, is set up. The QA device is exposed to a radiation field with the collimator at an angle. The exposure to radiation field is repeated for various collimator angles. A center of mass of all of the radiation fields imaged by the QA device gives a measure of the isocenter integrity of collimator rotation. FIG. 35 illustrates an exemplary exposure of the QA device at an angle of collimator rotation in order to assess integrity of collimator rotation, and also an exemplary range of rotation of the collimator disposed around the exemplary exposure. The center of mass can be analyzed digitally using a computer, table computing device, smart phone, server, or any other suitable digital processing unit or computing device. Alternately the center of mass can be analyzed manually.

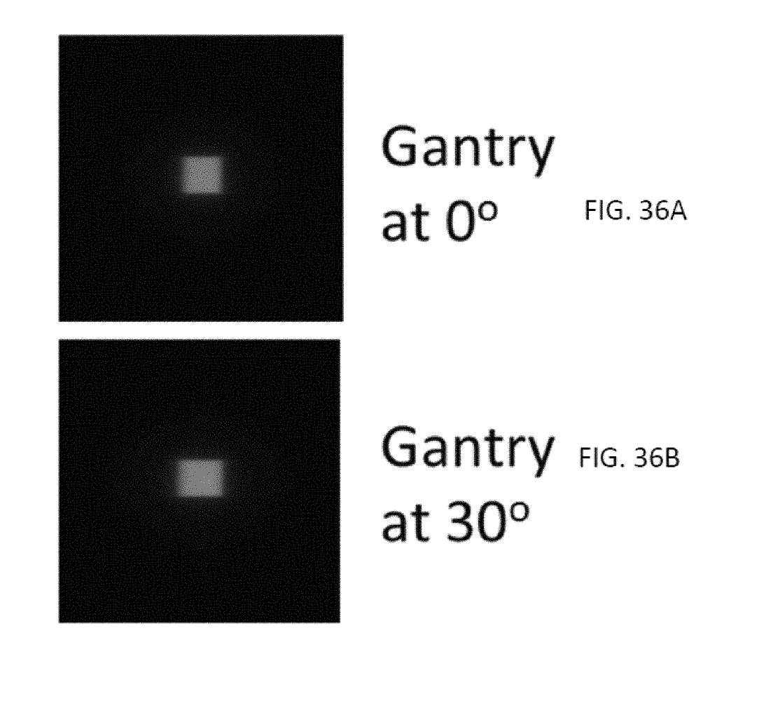

Another method in accordance with the present invention includes using a QA device of the present invention, described above, to assess the integrity of gantry rotation. The method includes setting the QA device flat at the isocenter of the gantry and/or the couch. Preferably, the QA device is positioned such that it hangs off of an end of the couch. A small square field of approximately 2 cm×2 cm, or any other suitable field size known to or conceivable by one of skill in the art, is set up. The QA device is exposed to the radiation field with the gantry angled at +/−60° above the QA device. A square radiation field image will become rectangular at certain gantry angles. The box can then be rotated 90°, 180°, and 270°. The center of mass of all of the radiation fields will give a measure of the integrity of the isocenter of the gantry rotation in a top half of the gantry space. The center of mass can be analyzed digitally using a computer, table computing device, smart phone, server, or any other suitable digital processing unit or computing device. Alternately the center of mass can be analyzed manually. FIG. 36A illustrates an exemplary exposure of the QA device at a 0° angle of gantry rotation in order to assess integrity of gantry rotation. FIG. 36B illustrates an exemplary exposure of the QA device at a 30° angle of gantry rotation in order to assess integrity of gantry rotation.

Yet another method of using the QA device described herein includes reconstructing the 3D map of the radiation treatment isocenter of a medical acceleration. For the delivery of high precision radiation treatment, the accuracy with which a target is irradiated at individual gantry, collimator and patient couch orientation is traditionally verified in 2D. With the QA device described herein, the coverage of the gantry is uniquely measured in 3D and, combining with the collimator and patient couch measurements, allows the reconstruction of the target coverage in full 3D, not possible before. In addition, the method of the present invention can be applied to decompose the traditional quality assurance measurements of combined gantry, collimator and patient couch orientations with standard devices so as to provide a comprehensive description of the irradiation accuracy.

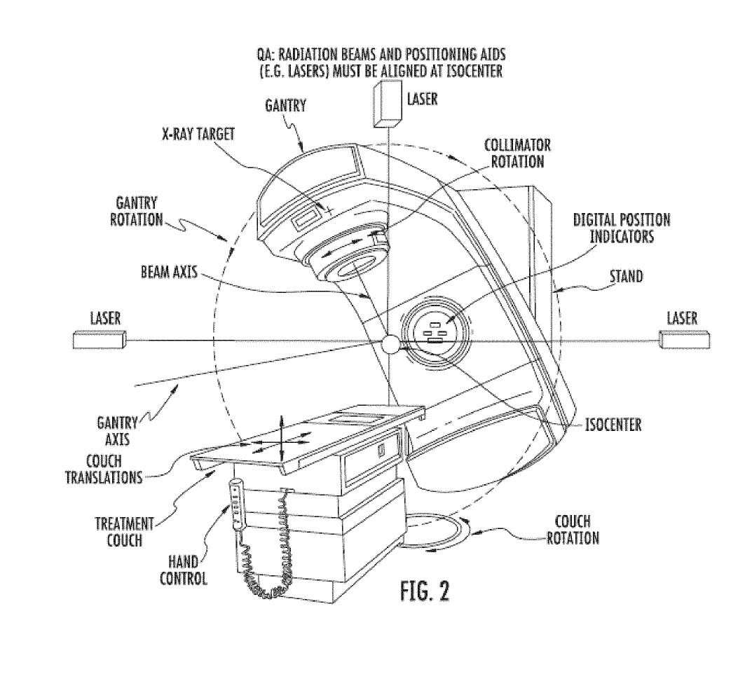

A medical accelerator, as shown in FIG. 2, is designed to direct a collimated radiation beam from any direction in the three-dimensional (3D) space to irradiate a target located at the treatment isocenter. This can be achieved by rotating the three mechanical components of the accelerator (the gantry, the collimator and the (patient) table) about the machine treatment “isocenter,” which is the interception of the rotation axes of three individual mechanical components. The accuracy and precision of these motions are imperative for accurate treatment and must be maintained. Quality assurance (QA) measurements are therefore frequently conducted to ensure that these motions are within specified limits.

In conventional practice, the collimator rotation is orthogonal to the (x,y) axis and is measured by placing a film laid flat in the (x,y) plane at the isocenter at z=0. The table rotation is also orthogonal to the (x,y) axis and therefore is also measured similarly with a flat film at the isocenter. A rotated gantry can pierce through at a point located at (x,y,z) on the isocenter axes. The measurement of the gantry (x,y,z) piercing point is difficult with any stationary 2D detector. Present practice employs a 2D film sandwiched between 2 vertical blocks in the y-z plane centered at isocenter so that irradiation by a small radiation beam at different gantry angle would form a “spoke” pattern, so-called a star-shot, in the y-z place. There is no information in the x-z plane as such an irradiation arrangement would only form spot on the film.

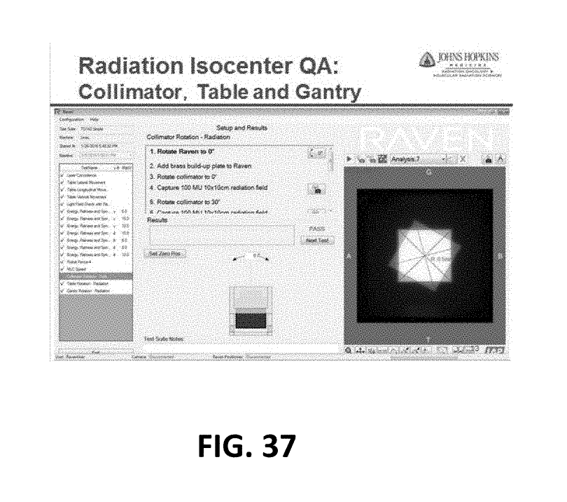

As described herein, the QA device provides a 2D measurement receptor that can rotate about the treatment isocenter. When the QA device is rotated orthogonal to the gantry beam, a transformation can be performed of the projected beam image to provide the piercing point of the beam in the (x, y, z) coordinates. FIG. 37 illustrates the user-interface of the Raven system to measure the rotation integrity of the gantry, collimator and table, according to an embodiment of the present invention. As illustrated in FIG. 37, the rotation of the gantry, collimator and table can all be tracked by the QA device. The user is instructed on how to take these measurements with the QA device and the results are displayed on a display screen for the user. Results of the testing can be displayed for the user, in order to show errors in the alignment of the components of the machine and action items that need to be executed in order to recalibrate the machine.

By combining a multitude of table rotation measurements in the (x,y) coordinate and the transformed gantry rotation measurements, incorporating additional collimator rotations at each rotated gantry angle, in (x,y,z) coordinate, it is possible to produce a 3D map, or region, in space that describes the extent of the radiation treatment isocenter of the machine. This is a 3D QA parameter that can be uniquely provided by the QA device described herein.

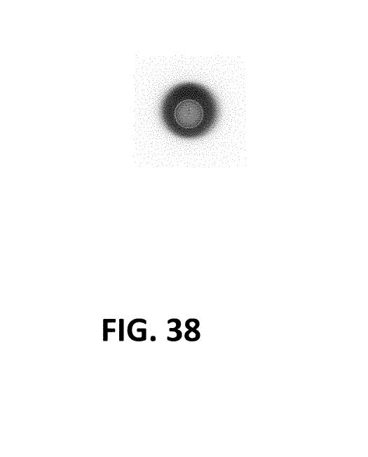

The QA device described herein can also be used to execute the Winston-Lutz (WL) test for radiosurgery QA. The WL is used by almost, if not, all practitioners to verify a patient's radiosurgery using a medical accelerator. The measurements consist of securing an extended bb marker off the treatment couch in the room, and irradiating it with the treatment beam at a combination of 20 gantry, collimator and couch positions (rotations). Each irradiation is imaged with the flat panel imager, or a film, on the gantry (mounted on the opposite side of the accelerator head downstream of the bb). The images are then used to verify that the beam covers the bb for all combinations of the machine orientation; after which patient treatment proceeds. FIG. 38 illustrates an image view of an irradiated WL bb, according to current practice of radiosurgery QA. FIG. 38 shows such an image of irradiated WL bb at a certain gantry, collimator and table rotation.

The individual measurements of independent gantry, collimator and table, or independent table and combined gantry and collimator, using the present invention can also be transformed and recombined to give results equivalent to WL test image, describing the displacement between the actual beam center and an idealized “point” radiation isocenter. Results can be displayed for the user, in order to show errors in the alignment of the components of the machine and action items that need to be executed in order to recalibrate the machine.

As a corollary, using the approach of measuring the WL images of the rotation of individual mechanical components (gantry, collimator and table), it is also possible to reconstruct from these “de-composed” images into an image of any combination of the individually measured components. Therefore, if 5 table, 5 gantry and 5 collimator measurements are completed, 15 measurements only, 125 combinations of WL tests are produced, rather than the current 20 measurements. To simplify analysis, it may be desirable to obtain 15 measurements of combined gantry and collimator rotations and 5 measurements ff independent table rotations, the resultant 20 measurements can still be used to yield 75 combinations of WL tests. It should be noted that while this method can be used with the present QA device invention described herein, it can also be applied with any other suitable QA device known to or conceivable by one of skill in the art.

The present invention can be carried out with a computer, non-transitory computer readable medium, or alternately a computing device or non-transitory computer readable medium incorporated into the scanner, QA device, or a standalone device. Indeed, any suitable method of calculation known to or conceivable by one of skill in the art could be used. It should also be noted that while specific measurement procedures and analysis are detailed herein, variations on these procedures and analysis can also be derived, and this application includes any such equation known to or conceivable by one of skill in the art.

A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape.

The computing device can be a special computer designed specifically for this purpose. The computing device can be unique to the present invention and designed specifically to carry out the method of the present invention. Scanners generally have a console which is a proprietary master control center of the scanner designed specifically to carry out the operations of the scanner and receive the imaging data created by the scanner. Typically, this console is made up of a specialized computer, custom keyboard, and multiple monitors. There can be two different types of control consoles, one used by the scanner operator and the other used by the physician. The operator's console controls such variables as the thickness of the image, the amount of tube current/voltage, mechanical movement of the patient table and other radiographic technique factors. The physician's viewing console allows viewing of the images without interfering with the normal scanner operation. This console is capable of rudimentary image analysis. The operating console computer is a non-generic computer specifically designed by the scanner manufacturer for bilateral (input output) communication with the scanner. It is not a standard business or personal computer that can be purchased at a local store. Additionally this console computer carries out communications with the scanner through the execution of proprietary custom built software that is designed and written by the scanner manufacturer for the computer hardware to specifically operate the scanner hardware.

Although the present invention has been described in connection with preferred embodiments thereof, it will be appreciated by those skilled in the art that additions, deletions, modifications, and substitutions not specifically described may be made without departing from the spirit and scope of the invention as defined in the appended claims.

1. A method for real-time mechanical and dosimetric quality assurance measurements in radiation therapy, comprising:
obtaining measurements of an orientation of a component of the medical accelerator in two dimensions using a quality assurance (QA) device at an isocenter of a medical accelerator;
measuring coverage of the gantry in three dimensions using a QA device at an isocenter of a medical accelerator; and
reconstructing and displaying target coverage of the gantry in three dimensions.

2. The method of claim 1 further comprising the component of the medical accelerator comprises the collimator, the table/couch, and the gantry.

3. The method of claim 1 further comprising rotating the QA device about a treatment isocenter of the medical accelerator.

4. The method of claim 1 further comprising rotating the QA device orthogonal to a gantry beam such that a transformation can be performed on the projected beam image.

5. The method of claim 4 further comprising providing a piercing point of the gantry beam in (x, y, z) or three dimensional coordinates.

6. The method of claim 1 further comprising displaying results of the measurements taken with the QA device.

7. The method of claim 1 further comprising displaying errors in alignment of components of the medical accelerator based on the measurements taken with the QA device.

8. A method for verifying a patient's radiosurgery, comprising:
securing an extended bb marker off of a treatment couch in a treatment room;
irradiating the extended bb marker with a treatment beam from a medical accelerator;
imaging the irradiation with a flat panel imager;
verifying that the treatment beam covers the bb for all combinations of orientation of the medical accelerator; and
reconstructing and displaying an image of any combination of rotation of a component of the medical accelerator.

9. The method of claim 8 further comprising irradiating the extended bb marker at a number of orientations of a gantry, collimator, and couch of the medical accelerator.

10. The method of claim 9 further comprising performing 15 to 20 irradiations of the extended bb marker at different orientations of the gantry, collimator, and couch of the medical accelerator.

11. The method of claim 8 further comprising calibrating the medical accelerator based on the outcome of the irradiating.

12. The method of claim 8 further comprising positioning the flat panel imager on a gantry on an opposite side of the accelerator head.

13. The method of claim 8 further comprising reconstructing an image of a gantry, collimator, and couch of the medical accelerator.

14. The method of claim 8 further comprising calculating displacement between the actual beam center and an idealized “point” radiation isocenter.

15. A method for real-time mechanical and dosimetric quality assurance measurements in radiation therapy, comprising:
obtaining measurements of an orientation of a component of the medical accelerator in two dimensions using a quality assurance (QA) device at an isocenter of a medical accelerator;
measuring coverage of the gantry in three dimensions using a QA device at an isocenter of a medical accelerator;
reconstructing target coverage of the gantry in three dimensions; and
calculating displacement between the actual beam center and an idealized “point” radiation isocenter

16. The method of claim 15 further comprising irradiating the QA device at a number of orientations of a gantry, collimator, and couch of the medical accelerator.

17. The method of claim 16 further comprising performing 15 to 20 irradiations of the QA device at different orientations of the gantry, collimator, and couch of the medical accelerator.

18. The method of claim 15 further comprising calibrating the medical accelerator based on the outcome of the irradiating.

19. The method of claim 15 further comprising positioning the QA device on a gantry on an opposite side of the accelerator head.

20. The method of claim 15 further comprising reconstructing and displaying an image of a gantry, collimator, and couch of the medical accelerator.
